(19) United States
(12) Patent Application Publication
(10) Pub. No.: US 2025/0186326 A1
(43) Pub. Date: Jun. 12, 2025

(54) Anti-Aging Compositions

(71) Applicant: PROGELIFE, Marseille (FR)

(72) Inventors: Sophie PERRIN, Marseille (FR); Claire NAVARRO, Marseille (FR); Héloïse LAROYE, Marseille (FR); Eric DESSAUD, Marseille (FR); Pierre CAU, Marseille (FR)

(21) Appl. No.: 18/846,212

(22) PCT Filed: Mar. 11, 2023
(86) PCT No.: PCT/EP2023/056256

§ 371 Date: Sep. 11, 2024

(30) Foreign Application Priority Data
Mar. 11, 2022 (EP) 22315060.8

(51) Int. Cl. A61K 8/60; A61K 8/35; A61K 8/49; A61Q 19/08

(52) U.S. Cl. CPC A61K 8/602; A61K 8/35; A61K 8/498; A61Q 19/08; A61K 2800/92

(57) ABSTRACT

The present invention relates to a composition comprising at least one first compound of the formula (I), or a polymer of the first compound and at least one second compound of formula (I), of the following formula (II) or of the following formula (III), the second compound of formula (I) being different from the first compound of formula (I).

### FIELD OF THE INVENTION

The present invention relates to a composition useful as a cosmetic agent or a food supplement, in particular an anti-aging or antioxidant cosmetic agent or food supplement.

### TECHNICAL BACKGROUND

Finding solutions to preserve a youthful physical appearance in spite of aging has been a constant preoccupation throughout human history. However, it is only recently that anti-aging cosmetic active ingredients having a demonstrated effect of reducing the visible signs of aging, instead of simply camouflaging them, have been discovered.

Among these actives, peptides are particularly successful in the current anti-aging market, and there are increasing numbers of double-blinded, placebo-controlled studies establishing their effects on human skin.

By way of example, the palmitoyl pentapeptide palmitoyl-lysine-threonine-threonine-lysine-serine (pal-KTTKS) is a synthetic material that was designed as a topical agent to stimulate collagen production and thus to provide a skin anti-wrinkle benefit. According to a 12-week, double-blind, placebo-controlled, split-face, left-right randomized clinical study assessing two topical products: moisturizer control product vs. the same moisturizer product containing 3 ppm pal-KTTKS, pal-KTTKS provided significant improvement vs. placebo control for reduction in wrinkles/fine lines by both quantitative technical and expert grader image analysis (Robinson et al. (2005) International Journal of Cosmetic Science 27:155-160).

However, the cost of these peptides remains a challenge, especially if the peptide has low potency and requires greater concentrations to achieve efficacy. Besides, there is also a growing demand from consumers for cosmetic or nutrition products which are not only efficient but also of non-synthetic origin.

Accordingly, there is still a need for alternative anti-aging active ingredients suitable for cosmetic or nutrition use.

### SUMMARY OF THE INVENTION

The present invention arises from the unexpected finding, by the inventors, that the combination of compounds of formulae I.1a, I.4a, I.6a, I.7a and I.8a with compounds of formulae I.12, II.1, II.2, II.3a and II.5 could inhibit farnesylation of prelamin A, a surrogate marker of progerin accumulation, and hence have an anti-aging and antioxidant effects, therefore alleviating other age-associated metabolic defects. The inventors have also shown that a combination of compounds I.1a, II.1 and II.3a had a synergistic effect on the increase of the LC3II/I ratio and on the increase of the amount of Lamin B1 in human skin fibroblasts, thereby evidencing a synergistic anti-aging effect of the combination.

The present invention thus relates to a composition comprising at least one first compound of the following formula (I), or a polymer of the first compound:

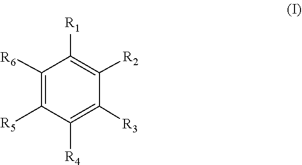

wherein:
   R₁ and R₄, identical or different, represent —H, —OH, or —OCH₃,
   R₂ and R₃, identical or different, represent —H, —OH, —OSO₃H, —O-hexoside, or —O—R₃₁, —O—CO—R₃₁ or —CH₂O—CO—R₃₁, wherein R₃₁ represents a saturated or unsaturated, linear or branched, aliphatic group comprising from 1 to 30 carbon atoms, optionally substituted by at least one ═O, —OH or —COOH group, or R₂ and R₃ represent together a group of the following formula (Ia) or (Ib):

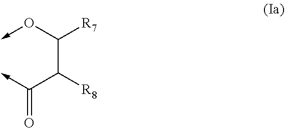

wherein:
R₇ represents —H, —OH, —OCH₃ or an aryl group comprising from 5 to 8 carbon atoms optionally substituted by at least one-OH group or oxyalkyl group comprising from 1 to 5 carbon atoms,
R₈ represents —H, —OH or —OCH₃,

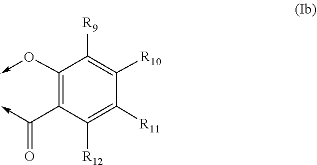

wherein R₉, R₁₀, R₁₁ et R₁₂, identical or different, represent —H, —OH or —OCH₃,
one of R₅ and R₆ represents —H, —OH or —OCH₃ and the other represents a saturated or unsaturated, linear or branched, aliphatic group comprising from 1 to 10 carbon atoms, optionally substituted by at least one ═O, —OH or —COOH group, or a group of the following formula (Ic):

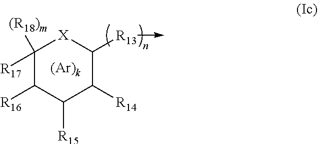

wherein
n=0 or 1,
k=0 or 1, when k=1 the above cycle is aromatic (Ar) and when k=0 the above cycle is not aromatic,
m=1 when k=0 and m=0 when k=1,
X represents —O—, —CH— or —CH₂—,
R₁₃ represents —O—, a group R₂₃ or a group —O—R₂₃— wherein R₂₃ represents a saturated or unsaturated, linear or branched, aliphatic group comprising from 1 to 5 carbon atoms, optionally substituted by at least one ═O group,
R₁₄ represents —H, —OH, —OCH₃ or a group of the following formula (Id):

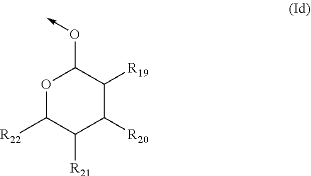

wherein R₁₉, R₂₀, R₂₁ and R₂₂, identical or different, represent —OH, —CH₂OH, an alkyl group comprising from 1 to 5 carbon atoms or an oxyalkyl group comprising from 1 to 5 carbon atoms,
R₁₅, R₁₆, R₁₇ and R₁₈, identical or different, represent —H, —OH, —OSO₃H, —CH₂OH, —COOH, —O-glucuronide, —O—R₃₀, —O—CO—R₃₀ or —CH₂O—CO—R₃₀, wherein R₃₀ represent a saturated or unsaturated, linear or branched, aliphatic group comprising from 1 to 30 carbon atoms, optionally substituted by at least one ═O, —OH or —COOH group,
and at least one second compound of formula (I), of the following formula (II) or of the following formula (III), the second compound of formula (I) being different from the first compound of formula (I):

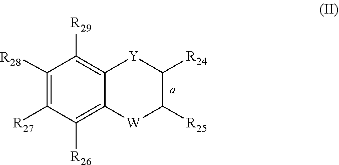

wherein:
   a represents a single bond or a double bond,
   Y represents —O— or —CO—,
   W represents —CH₂— or —CO—,
   R₂₄ represents an aryl group comprising from 5 to 8 carbon atoms, optionally substituted by at least one-OH, —OCH₃, —O-hexoside or —O-glucuronide group, R₂₅ represents —H, —OH, or an oxycarbonylaryl group comprising from 6 to 9 carbon atoms, optionally substituted by at least one-OH, —OCH₃, —O-hexoside or O-glucuronide group, or R₂₄ and R₂₅ represent together, with the carbon atoms to which they are attached and a representing a double bond, a aryl group comprising 6 carbon atoms, optionally substituted by at least one-OH or —CH₂OH group,
   R₂₆, R₂₇ and R₂₈, identical or different, represent —H, —OH, —OCH₃, —O-hexoside or O-glucuronide,
   R₂₉ represents —H, —OH or a group of the following formula (IIa):

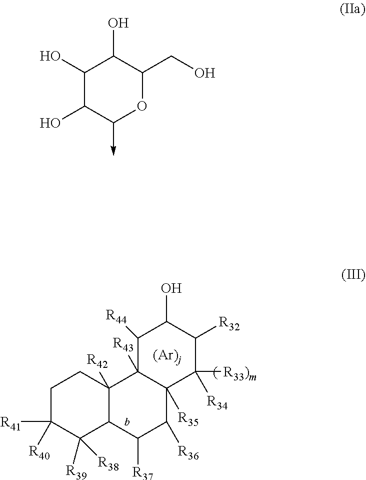

wherein:
   j=0 or 1, when j=1 the above cycle is aromatic (Ar) and when j=0 the above cycle is not aromatic,
m=1 when j=0 and m=0 when j=1,
   when j=1, R₃₂ represents an alkyl group having from 1 to 5 carbon atoms and when j=0, R₃₂ together with R₃₃ represent a group of the following formula (IIIa):

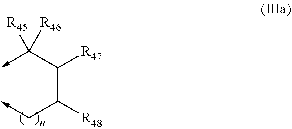

wherein:
n=0 or 1,
R₄₅ represents —H or —CH₃,
R₄₆ represents —CH₂OH or a group of the following formula (IIIb) or (IIIc):

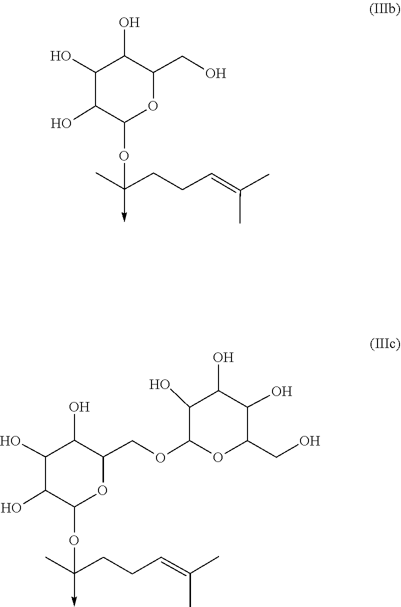

R₄₇ and R₄₈, identical or different, represent —H or —OH
R₃₄, R₃₅, R₃₈, and R₄₃, identical or different, represent —H or —CH₃,
R₃₆ represents —H, R₄₂ represents —CH₃, or together R₃₆ and R₄₂ represent a group of formula —O—CO—,
R₃₇ represents —H or —O-hexoside,
b represents a single bond or a double bond,
R₃₉ represents —CH₃, R₄₀ represents —H, or together R₃₉ and R₄₀ represent a group of the following formula (IIId):

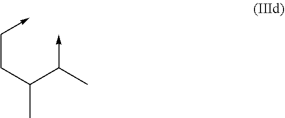

R₄₁ represents —H, —OH or a group of the following formula (IIIe) or (IIIf):

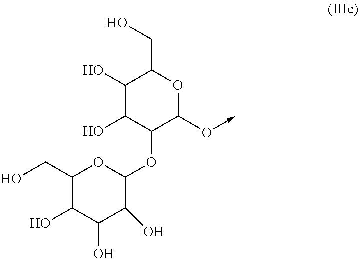

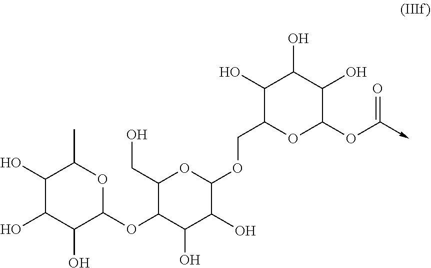

R₄₄ represents —H or —OH,
or cosmetically or nutritionally acceptable salts or esters thereof.

In an embodiment of the invention, the composition as defined above further comprises at least one third compound of formula (I), (II) or (III), provided the third compound is different from the first and second compounds.

The present invention also relates to the use of a composition as defined above, as a cosmetic agent or a human or animal food supplement, in particular an anti-aging or antioxidant cosmetic agent or food supplement.

The present invention also relates to the cosmetic use of a composition as defined above, to alleviate at least one sign of skin aging.

The present invention also relates to the use as cosmetic agents or food supplements, in particular as anti-aging or antioxidant cosmetic agents or food supplements, of at least one first compound of formula (I) as defined above, or of at least one polymer of the first compound, in combination with at least one second compound of formula (I), of formula (II) as defined above, or of formula (III) as defined above, the second compound of formula (I) being different from the first compound of formula (I).

The present invention also relates to the cosmetic use of at least one first compound of formula (I) as defined above, or of at least one polymer of the first compound, in combination with at least one second compound of formula (I), of formula (II) as defined above, or of formula (III) as defined above, the second compound of formula (I) being different from the first compound of formula (I), to alleviate at least one sign of skin aging.

### DETAILED DESCRIPTION OF THE INVENTION

As intended herein, the word “comprising” is synonymous to “include” or “contain”. When a subject-matter is said to comprise one or several features, it is meant that other features than those mentioned can be comprised in the subject-matter. Conversely, the expression “constituted of” is synonymous to “consisting of”. When a subject-matter is said to consist of one or several features, it is meant that no other features than those mentioned are comprised in the subject-matter.

As should be clear to one of skill in the art, all the stereochemical configurations of the compounds according to the invention are intended to be covered by the formulae shown herein. In particular, as is intended herein, when the stereoconfiguration of a bond is not specified, the bond may represent any of an upward bond, a downward bond, and a mixture of the two, in particular a 1/1 mixture of the two.

In the formulae described herein, an arrow represents the bond by which a group is attached to a compound comprising it.

As intended herein an —O-hexoside group represents an hexose group which is bound by an —O— bond. By way of example an —O-hexoside group according to the invention is represented by the following formula:

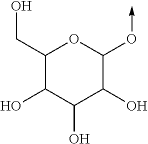

Preferably, the —O-hexoside group is an —O-glucoside group. Preferably an —O-glucoside group is represented by one of the following formulae:

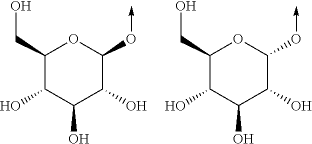

As intended herein an —O-glucuronide group is represented by one of the following formulae:

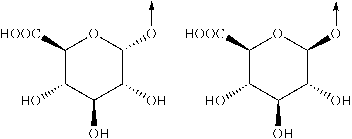

As intended herein, an oxyalkyl group can be represented as an —O-alkyl group.

Preferred alkyl groups according to the invention encompass the methyl, ethyl, n-propyl, isopropyl, n-butyl, s-butyl and t-butyl groups.

As intended herein, an oxycarbonylaryl group can be represented as an —O—CO-aryl group where CO represents C═O.

As intended herein “cosmetically or nutritionally acceptable salts or esters” are salts or esters of the compounds of formula (I), (II) or (III) as defined above which are suitable for a cosmetic or nutritional use.

Preferred cosmetically or nutritionally acceptable salts according to the invention are selected from the group consisting of hydrochloride, chloride, hydrobromide, bromide, iodide, phosphate/diphosphate, sulfate, tartrate, citrate, maleate, acetate, mesylate, pamoate (embonate), sodium, potassium, calcium, meglumine, zinc, lithium, and magnesium.

Preferred cosmetically or nutritionally acceptable esters according to the invention are formed between at least one hydroxyl (—OH) group of a compound of formula (I), (II) or (III) according to the invention and at least one carboxylic acid of formula HOOC—R, wherein R represent a saturated or unsaturated, linear or branched, aliphatic group comprising from 1 to 30 carbon atoms, optionally substituted by at least one ═O, —OH or —COOH group.

### First Compound

Preferably, the first compound is selected from the group consisting of the compounds of the following formulae:

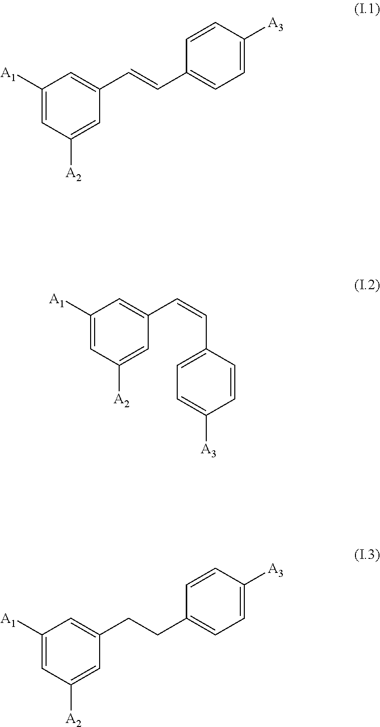

wherein A₁, A₂ and A₃, identical or different, represent —OH, —OSO₃H, —O-glucuronide, or —O—CO-A₄, wherein A₄ represents an aliphatic group comprising from 1 to 30 carbon atoms, saturated or unsaturated, linear or branched, optionally substituted by at least one ═O, —OH or —COOH group,

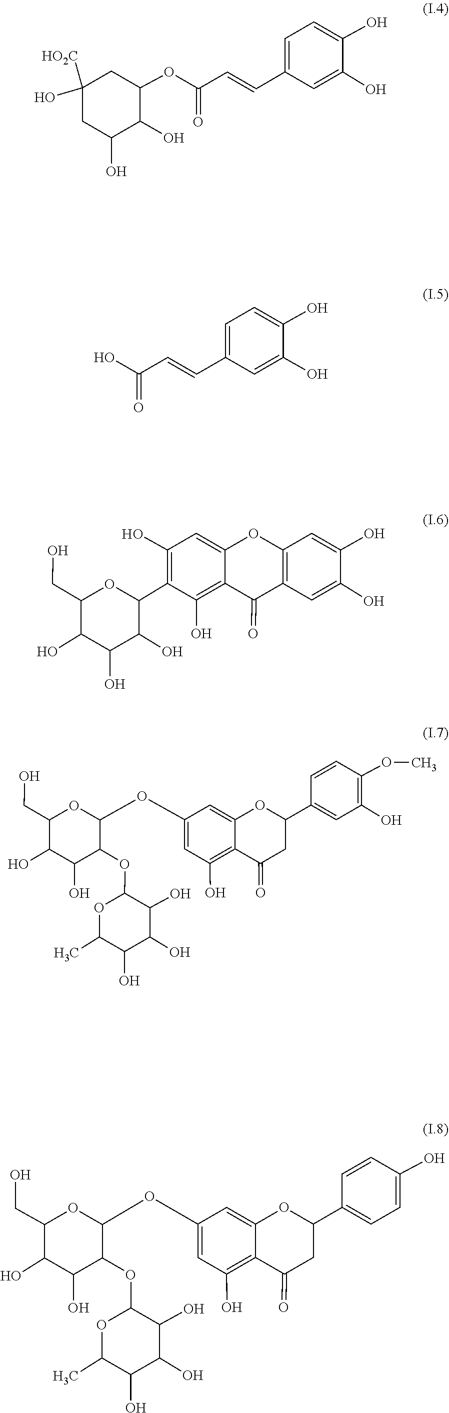 [SMILES: O=C(/C=C/c1ccc(O)c(O)c1)OC1CC(O)(C(=O)O)CC(O)C1O ; O=C(O)/C=C/c1ccc(O)c(O)c1 ; O=c1c2cc(O)c(O)cc2oc2cc(O)c(C3OC(CO)C(O)C(O)C3O)c(O)c12 ; COc1ccc(C2CC(=O)c3c(O)cc(OC4OC(CO)C(O)C(O)C4OC4OC(C)C(O)C(O)C4O)cc3O2)cc1O ; CC1OC(OC2C(Oc3cc(O)c4c(c3)OC(c3ccc(O)cc3)CC4=O)OC(CO)C(O)C2O)C(O)C(O)C1O]

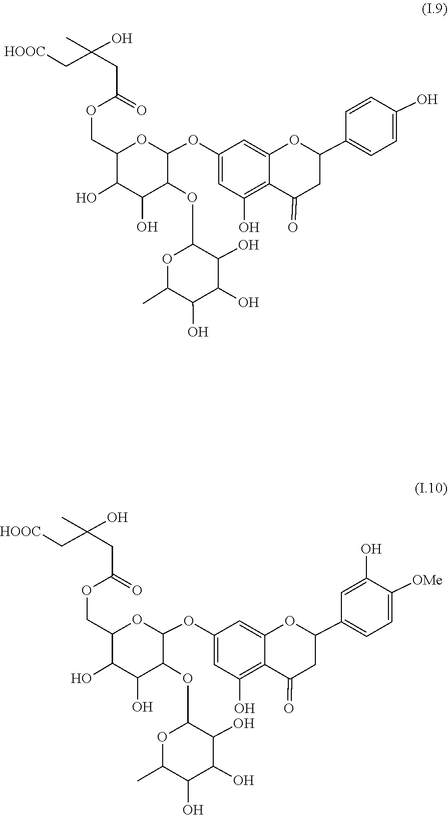 [SMILES: CC1OC(OC2C(Oc3cc(O)c4c(c3)OC(c3ccc(O)cc3)CC4=O)OC(COC(=O)CC(C)(O)CC(=O)O)C(O)C2O)C(O)C(O)C1O ; COc1ccc(C2CC(=O)c3c(O)cc(OC4OC(COC(=O)CC(C)(O)CC(=O)O)C(O)C(O)C4OC4OC(C)C(O)C(O)C4O)cc3O2)cc1O]

Preferably, the compound of the above formula (I.1) has one of the following combinations of substituents:

| A₁ | A₂ | A₃ | CAS number |
|---|---|---|---|
| —OH | —OH | —OH | 501-36-0 |
| sulfate | —OH | sulfate | 858187-20-9 |
| sulfate | sulfate | —OH | 858187-21-0 |
| —O-glucuronide | —OH | —OH | 387372-17-0 |
| sulfate | —OH | —OH | 553662-69-4 |
| —OH | —OH | —O-glucuronide | 387372-20-5 |
| —OH | —OH | sulfate | 858187-19-6 |

Preferably, the compound of the above formula (I.2) has one of the following combinations of substituents:

| A₁ | A₂ | A₃ | CAS number |
|---|---|---|---|
| —OH | —OH | —OH | 61434-67-1 |
| —O-glucuronide | —OH | —OH | 387372-23-8 |
| sulfate | —OH | —OH | —* |
| —OH | —OH | —O-glucuronide | 387372-26-1 |
| —OH | —OH | sulfate | —** |

*[3-hydroxy-5-[(Z)-2-(4-hydroxyphenyl)ethenyl]phenyl] hydrogen sulfate
**[4-[(Z)-2-(3,5-dihydroxyphenyl)ethenyl]phenyl] hydrogen sulfate

Preferably, the compound the compound of the above formula (I.3) has one of the following combinations of substituents:

| A₁ | A₂ | A₃ | CAS number |
|---|---|---|---|
| —OH | —OH | —OH | 58436-28-5 |

Polymers of the first compounds according to the invention are preferably polymers of compounds of formula (I.1), (I.2) or (I.3), in particular as described in International application WO99/03816, which is incorporated herein by reference.

Preferably, the compound of formula (I.4) is represented by the following formula (I.4a) (CAS number 327-97-9):

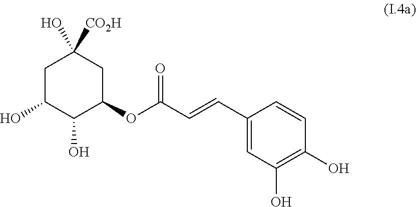 [SMILES: O=C(/C=C/c1ccc(O)c(O)c1)O[C@@H]1C[C@](O)(C(=O)O)C[C@@H](O)[C@H]1O]

Preferably, the compound of formula (I.6) is represented by the following formula (I.6a) (CAS number 4773-96-0):

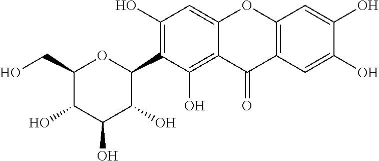 [SMILES: O=c1c2cc(O)c(O)cc2oc2cc(O)c([C@@H]3O[C@H](CO)[C@@H](O)[C@H](O)[C@H]3O)c(O)c12]

Preferably, the compound of formula (I.7) is represented by the following formula (I.7a) (CAS number 13241-33-3):

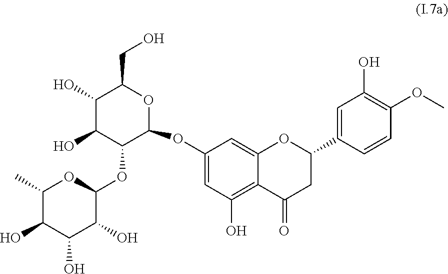

Preferably, the compound of formula (I.8) is represented by the following formula (I.8a) (CAS number 10236-47-2):

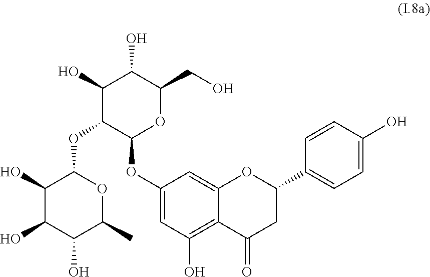 [SMILES: C[C@@H]1O[C@@H](O[C@H]2[C@H](Oc3cc(O)c4c(c3)O[C@H](c3ccc(O)cc3)CC4=O)O[C@H](CO)[C@@H](O)[C@@H]2O)[C@H](O)[C@H](O)[C@H]1O]

### Second Compound

Preferably, the second compound is selected from the group consisting of the compounds of the following formulae:

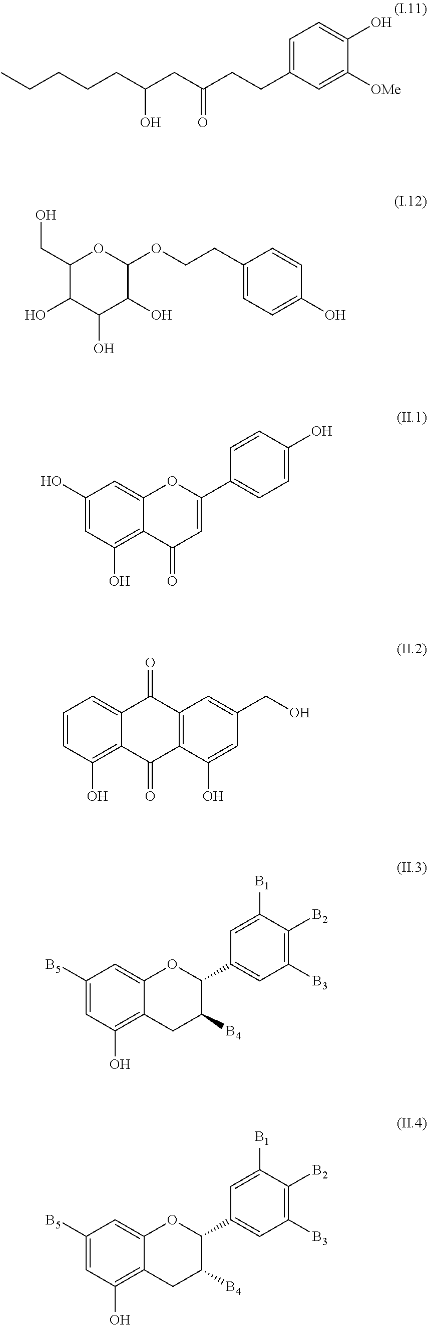

wherein:
   B₁ represents —OH, —OCH₃ or —O-glucuronide,
   B₂ represents —OH or —OCH₃,
   B₃ represents —H or —OH,
   B₄ represents —OH, —O-glucuronide or a group of the following formula:

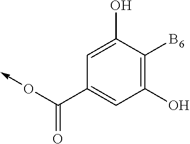

wherein B₆ represents —OH, —OCH₃ or —O-glucuronide,
B₅ represents —OH, —O-hexoside or —O-glucuronide,

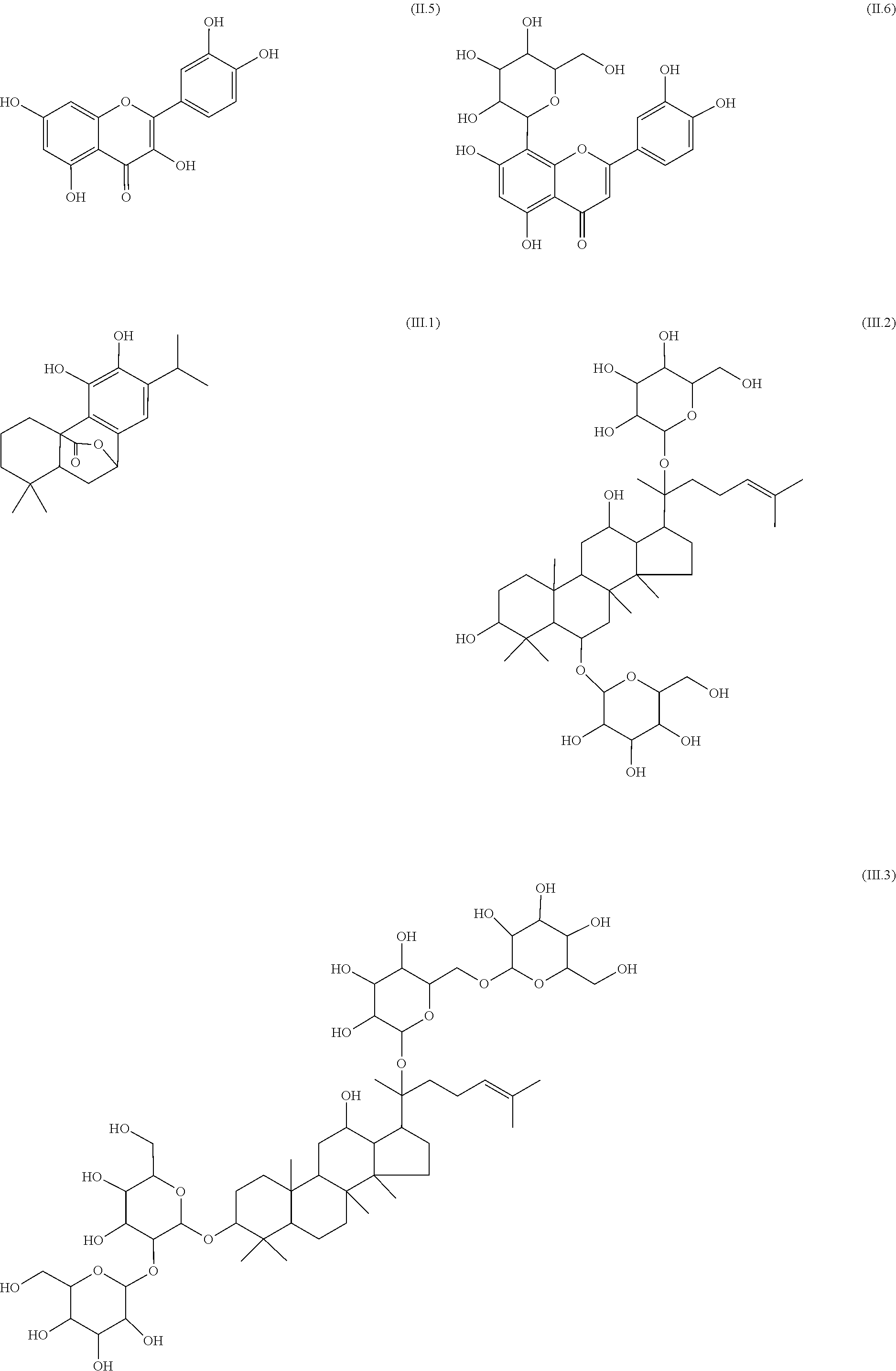 [SMILES: O=c1c(O)c(-c2ccc(O)c(O)c2)oc2cc(O)cc(O)c12 ; O=c1cc(-c2ccc(O)c(O)c2)oc2c(C3OC(CO)C(O)C(O)C3O)c(O)cc(O)c12 ; CC(C)c1cc2c(c(O)c1O)C13CCCC(C)(C)C1CC2OC3=O ; CC(C)=CCCC(C)(OC1OC(CO)C(O)C(O)C1O)C1CCC2(C)C1C(O)CC1C3(C)CCC(O)C(C)(C)C3C(OC3OC(CO)C(O)C(O)C3O)CC12C ; CC(C)=CCCC(C)(OC1OC(COC2OC(CO)C(O)C(O)C2O)C(O)C(O)C1O)C1CCC2(C)C1C(O)CC1C3(C)CCC(OC4OC(CO)C(O)C(O)C4OC4OC(CO)C(O)C(O)C4O)C(C)(C)C3CCC12C]

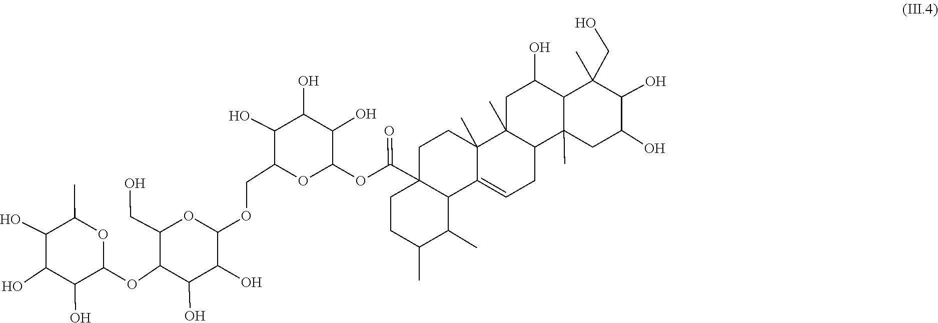 [SMILES: CC1CCC2(C(=O)OC3OC(COC4OC(CO)C(OC5OC(C)C(O)C(O)C5O)C(O)C4O)C(O)C(O)C3O)CCC3(C)C(=CCC4C5(C)CC(O)C(O)C(C)(CO)C5C(O)CC43C)C2C1C]

Preferably, the compound of the above formula (I.12) is represented by the following formula (I.12a) (CAS number 10338-51-9):

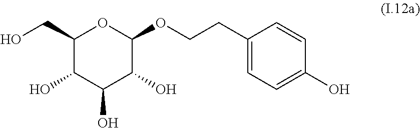 [SMILES: OC[C@H]1O[C@@H](OCCc2ccc(O)cc2)[C@H](O)[C@@H](O)[C@@H]1O]

Preferably, the compound of the above formula (II.3) has one of the following combinations of substituents:

| B₁ | B₂ | B₃ | B₄ | B₅ | B₆ | CAS number |
|---|---|---|---|---|---|---|
| —OH | —OH | —H | —OH | —OH | — | 154-23-4 |
| —OCH₃ | —OH | —H | —OH | —OH | — | 60383-97-3 |

Preferably, the compound of the above formula (II.3) is represented by the following formula (II.3a):

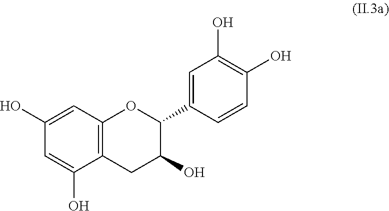 [SMILES: Oc1cc(O)c2c(c1)O[C@H](c1ccc(O)c(O)c1)[C@@H](O)C2]

Preferably, the compound of the above formula (II.4) has one of the following combinations of substituents:

| | | | | | | CAS |
|---|---|---|---|---|---|---|
| B₁ | B₂ | B₃ | B₄ | B₅ | B₆ | number |
| —OH | —OH | —H | —OH | —OH | — | 490-46-0 |
| —OH | —OH | —H | 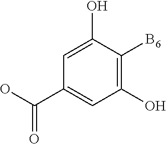 | —OH | —OH | 1257-08-5 |
| —OH | —OH | —OH | —OH | —OH | — | 970-74-1 |
| —O-glucoronide | —OH | —OH | —OH | —OH | — | 569670- |
| | | | | | | 41-3 |
| —OH | —OH | —OH | 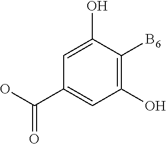 | —OH | —OH | 989-51-5 |
| —OH | —OH | —OH | —O-glucoronide | —OH | — | —* |
| —OH | —OH | —OH | —OH | —O-glucoronide | — | 569670- |
| | | | | | | 42-4 |
| —OCH₃ | —OH | —H | —OH | —O-glucoronide | — | 603133- |
| | | | | | | 94-4 |
| —OCH₃ | —OH | —H | —OH | —OH | — | 76549-34- |
| | | | | | | 3 |
| —OH | —OH | —OH | 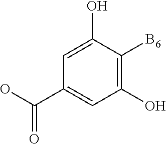 | —OH | —OCH₃ | 224434- 07-5 |
| —OH | —OCH₃ | —OH | 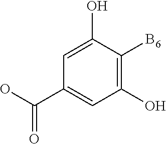 | —OH | —OCH₃ | 298700- 58-0 |
| —O-glucoronide | —OCH₃ | —H | —OH | —OH | — | —** |
| —O-glucoronide | —OCH₃ | —OH | —OH | —OH | — | 1038922- |
| | | | | | | 75-6 |
| —OH | —OCH₃ | —OH | —OH | —O-glucuronide | — | 1038922- |
| | | | | | | 74-5 |
| —OH | —OCH₃ | —H | —OH | —OH | — | 97914-20- |
| | | | | | | 0 |
| —OH | —OCH₃ | —OH | —OH | —OH | — | 17291-05- |
| | | | | | | 3 |
| —O-glucuronide | —OH | —H | —OH | —OH | — | 603133- |
| | | | | | | 91-1 |
| —OH | —OH | —H | —OH | —O-glucoronide | — | 389136- |
| | | | | | | 63-4 |
| —OH | —OH | —OH | 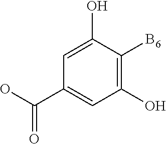 | —O-glucoside | —O- glucuro- nide | —*** |

*(2S,3S,4S,5R,6R)-6-[[(2R,3R)-5,7-dihydroxy-2-[3,4,5-trihydroxyphenyl)-3,4-dihydro-2H-chromen-3-yl]oxy]-3,4,5-trihydroxyoxane-2-carboxylic acid
**(2S,3S,4S,5R,6S)-3,4,5-trihydroxy-6-[2-methoxy-5-[(2R,3R)-3,5,7-trihydroxy-3,4-dihydro-2H-chromen-2-yl]phenoxy]oxane-2-carboxylic acid
***(2S,3S,4S,5R,6S)-6-[2,6-dihydroxy-4-[[(2R,3R)-5-hydroxy-7-[(2S,3R,4S,5S,6R)-3,4,5-trihydroxy-6-(hydroxymethyl)oxan-2-yl]oxy-2-(3,4,5-trihydroxyphenyl]-3,4-dihydro-2H-chromen-3-yl]oxycarbonyl]phenoxy]-3,4,5-trihydroxyoxane-2-carboxylic acid

Preferably, the compound of the above formula (II.6) is represented by the following formula (II.6a) (CAS number 28608-75-5):

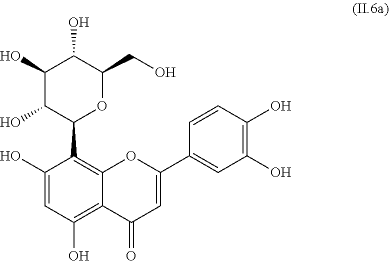 [SMILES: O=c1cc(-c2ccc(O)c(O)c2)oc2c([C@@H]3O[C@H](CO)[C@@H](O)[C@H](O)[C@H]3O)c(O)cc(O)c12]

Preferably, the compound of formula (III.2) is represented by the following formula (III.2a) (CAS number 41753-43-9):

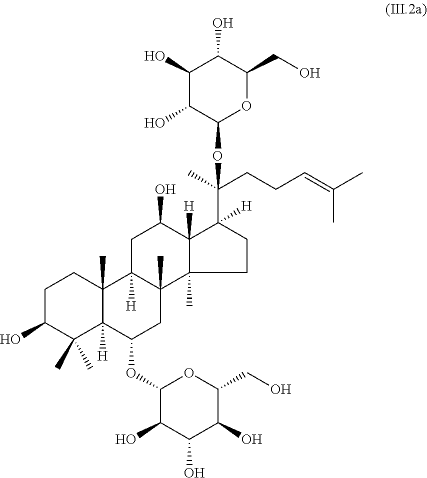

Preferably, the compound of the above formula (III.3) is represented by the following formula (III.3a) (CAS number 22427-39-0):

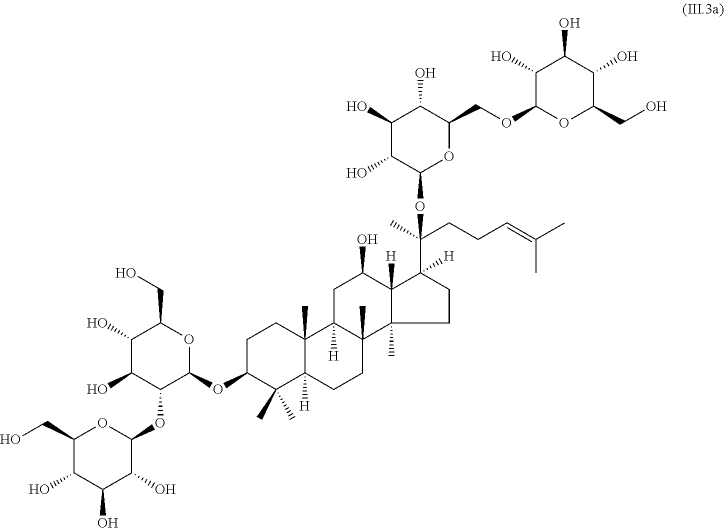

Preferably, the compound of formula (III.4) above is represented by the following formula (III.4a) (CAS number 34540-22-2):

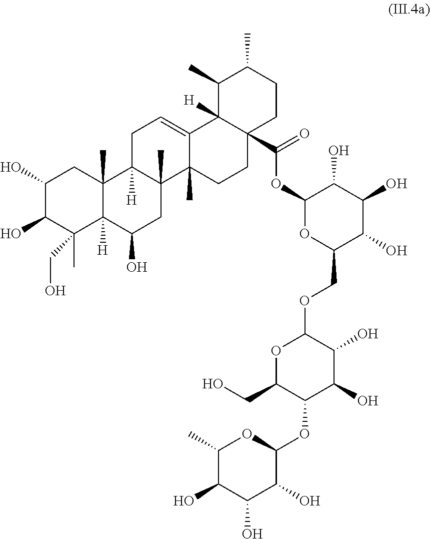

### Composition

Preferably, the composition as defined above comprises at least two different compounds selected from the following pairs of compounds:

TABLE A

| | I.11 | I.12 | II.1 | II.2 | II.3 | II.4 | II.5 | II.6 | III.1 | III.2 | III.3 | III.4 |
|---|---|---|---|---|---|---|---|---|---|---|---|---|
| I.1 | X | X | X | X | X | X | X | X | X | X | X | X |
| I.2 | X | X | X | X | X | X | X | X | X | X | X | X |
| I.3 | X | X | X | X | X | X | X | X | X | X | X | X |
| I.4 | X | X | X | X | X | X | X | X | X | X | X | X |
| I.5 | X | X | X | X | X | X | X | X | X | X | X | X |
| I.6 | X | X | X | X | X | X | X | X | X | X | X | X |
| I.7 | X | X | X | X | X | X | X | X | X | X | X | X |
| I.8 | X | X | X | X | X | X | X | X | X | X | X | X |
| I.9 | X | X | X | X | X | X | X | X | X | X | X | X |
| I.10 | X | X | X | X | X | X | X | X | X | X | X | X |

Preferably, the composition as defined above, in particular wherein the composition comprises at least two different compounds selected from the pairs defined in Table A, further comprises at least a third compound selected from the group consisting of the compounds of respective formulae I.1, I.2, I.3, I.4, I.5, I.6, I.7., I.8, I.9, I.10, I.11, I.12, II.1, II.2, II.3, II.4, II.5, II.6, III.1, III.2, III.3 and III.4, provided the third compound is different from the first and second compounds.

Preferably, the composition as defined above comprises at least two different compounds, or at least three different compounds, selected respectively from:
a compound of formula (I.1), (I.2) or (I.3),
a compound of formula (II.1), and
a compound of formula (II.3) or (II.4).

Most preferably, the composition as defined above comprises:
a compound of formula (I.1), (I.2) or (I.3),
a compound of formula (II.1), and
a compound of formula (II.3) or (II.4).

Even more preferably, the composition as defined above comprises:
a compound of formula (I.1), in particular of formula (I.1a);
a compound of formula (II.1), and
a compound of formula (II.3), in particular of formula (II.3a).

Preferably, the composition as defined above comprises:
a compound of formula (I.4), in particular of formula (I.4a);
a compound of formula (I.6), in particular of formula (I.6a), and
a compound of formula (II.1) or of formula (II.5).

Preferably, the composition as defined above comprises:
a compound of formula (I.4), in particular of formula (I.4a);
a compound of formula (II.1), and
a compound of formula (II.5).

Preferably, the composition as defined above comprises from 0.001 g to 5 g, more preferably from 0.01 g to 2.5 g, most preferably from 0.1 g to 1 g of the total weight of the compounds of formulae (I), (II) and/or (III) as defined above.

Preferably, the composition as defined above comprises from 0.01% (w/w) to 100% (w/w), more preferably from 0.05% (w/w) to 20% (w/w), most preferably from 0.1% (w/w) to 5% (w/w), of the total weight of the compounds of formulae (I), (II) and/or (III) as defined above, with respect to the total weight of the composition.

Preferably, the composition as defined above comprises from 70% (w/w) to 90% w/w) of a compound of formula (II.3) or (II.4) with respect to the total weight of the compounds of formulae (I), (II) and/or (III) as defined above in the composition.

Preferably, the composition as defined above comprises from 5% (w/w) to 15% w/w) of a compound of formula (I.1), (I.2) or (I.3) with respect to the total weight of the compounds of formulae (I), (II) and/or (III) as defined above in the composition.

Preferably, the composition as defined above comprises from 5% (w/w) to 15% w/w) of a compound of formula (II.1) with respect to the total weight of the compounds of formulae (I), (II) and/or (III) as defined above in the composition.

Most preferably, the composition as defined above comprises:
from 5% (w/w) to 15% (w/w), in particular from 8% (w/w) to 10% (w/w), of a compound of formula (I.1), (I.2) or (I.3)
from 5% (w/w) to 15% (w/w), in particular from 8% (w/w) to 10% (w/w), of a compound of formula (II.1), and
from 70% (w/w) to 90% (w/w), in particular from 80% (w/w) to 84% (w/w), of a compound of formula (II.3) or (II.4),
   with respect to the total weight of the compound of formula (I.1), (I.2) or (I.3), the compound of formula (II.1) and the compound of formula (II.3) or (II.4).

Even more preferably, the composition as defined above comprises:
from 5% (w/w) to 15% (w/w), in particular from 8% (w/w) to 10% (w/w), of a compound of formula (I.1), in particular of formula (I.1a)
from 5% (w/w) to 15% (w/w), in particular from 8% (w/w) to 10% (w/w), of a compound of formula (II.1), and
from 70% (w/w) to 90% (w/w), in particular from 80% (w/w) to 84% (w/w), of a compound of formula (II.3), in particular of formula (II.3a),
   with respect to the total weight of the compound of formula (I.1), the compound of formula (II.1) and the compound of formula (II.3).

Preferably, the compounds of formula (I), (II) and (III) as defined above are comprised in at least one vegetal extract.

As intended herein, the vegetal extract can be from a whole plant or from plant parts, such as leaves, flowers, fruits, zests, stems, bark, or roots, e.g. grape must. As intended herein, a vegetal extract can be obtained by applying to plant or plant parts at least one of grinding, pressing, infusing in an aqueous, e.g. water, or organic solvent, and/or passing an aqueous, e.g. water or organic solvent. Where a solvent is used, the solvent may be cold, e.g. from 10° C. to 30° C., or hot, e.g. from 50° C. to 200° C., liquid or gaseous. The vegetal extract can be a crude extract or a purified extract. The vegetal extract can be concentrated. In particular, the vegetal extract can be purified and/or concentrated to increase content in at least one compound of formula (I), (II) or (III) as defined above. Intended herein, the compounds of formula (I), (II) or (III) as defined above may be isolated and/or purified from a vegetal extract.

Preferably, the composition as defined above is a cosmetic composition.

Preferably, the composition as defined above is a nutritional composition or a nutritional or food supplement, in particular intended for human or animal consumption, i.e. a human or animal nutritional composition or nutritional/food supplement.

Preferably, the composition as defined above further comprises at least one cosmetically or nutritionally acceptable carrier or excipient.

As intended herein, a cosmetically acceptable carrier or excipient is compatible with the skin, it is preferably of pleasant colour, smell and consistency and does not generate discomfort such as prickling, redness or other discomfort likely to turn consumers away from use thereof.

A cosmetically acceptable carrier or excipient according to the invention notably includes diluting agent, dispersing agent, gelling agent, solid emollient, gums, resins, solvents, fillers such as modified and polymerised starches, or metal stearate, preserving agents, essential oils, pearling agents, colouring agents, odour absorbers, pH regulators or neutralising agents, thickening agents, absorption-promoting agents and in particular ethanol and/or phospholipids, flavouring or perfuming agents, mineral pigments such as iron oxides, oil agents such as oils or fat of vegetable origin, fats of animal origin, synthetic oils, silicone oils (cyclomethicone) fluorine-containing oils, fatty alcohol esters (cetyl alcohol), waxes, modified clays, bentones, fatty acid metal salts, hydrophobized silica, polyethylenes, mica and/or other substances used in cosmetics.

The cosmetic composition according to the invention can be a care or make-up product for example in the form of a serum, lotion, cream, milk, water or oil gel, hydrogel, mask, stick, patch, oil, unguent, wax, foam, toner, care solution, balm, foundation, spray, eye shadow, lipstick, paste, ointment, shampoo or conditioner.

Where the cosmetic composition according to the invention is intended for topical administration to the skin, it may be in the form of any preparation conventionally used for topical application. In particular, it may advantageously be in the form of aqueous solutions, hydroalcoholic solutions, oil-in-water emulsions (O/W) or water-in-oil emulsions (W/O) or multiple emulsions (triple: W/O/W or O/W/O), or micro- or nano-emulsions.

When the composition is in aqueous form, in particular in a dispersion, emulsion or aqueous solution, it may comprise an aqueous phase possibly containing water, flower water, fruit water and/or mineral water. Preferably, the fruit water is selected from the group consisting of lemon fruit water, orange fruit water, grapefruit fruit water, pomelo fruit water, apricot fruit water, tomato fruit water, mango fruit water, cucumber fruit water, pineapple fruit water, raspberry fruit water, tangerine fruit water, apple fruit water and blueberry fruit water.

The cosmetic composition of the invention may further comprise other active molecules such as vitamins, sunscreens and filters, anti-age active substances, anti-wrinkle agents in particular peptides, anti-oxidants, lightening agents, self-tanning ingredients, tanning accelerators, lifting ingredients, slimming agents, firming ingredients, hydrating ingredients, peeling ingredients, sebum-regulating agents, mattifying agents, etc. Preferably the cosmetic composition may comprise anti-age active substances, anti-wrinkle and/or anti-oxidant agents, a lifting ingredient, firming ingredient and/or hydrating ingredient. In general, persons skilled in the art are able to select and adapt the quantities of additional active molecules so that the properties of the composition of the invention are not deteriorated through the addition thereof.

As intended herein a nutritionally acceptable carrier or excipient relates to a food component suitable for human or animal consumption.

Preferably, the nutritionally acceptable carrier or excipient according to the invention is selected from the group consisting of anti-foaming agents, emulsifiers, firming agents, gelling agents, humectants, mineral salts, stabilizers, thickeners, and texturizing agent. Preferably, the anti-foaming agent is selected from the group consisting of polyethylene glycol and triethyl citrate. Preferably, the emulsifier is selected from lecithin, sorbitan monostearate and ammonium salts of phosphatidic acids. The firming agent is preferably selected from the group consisting of calcium chloride, calcium gluconate and calcium sulfate. The gelling agent is preferably selected from the group consisting of agar, calcium alginate and carrageenan. The humectant is preferably selected from the group consisting of glycerin, glycerol, lactitol and oxidized polyethylene; the mineral salt is cupric sulfate. Preferably, the stabilizer is selected from the group consisting of xanthan gum, guar gum and bleached starched. The thickener is preferably selected from the group consisting of tannins, sodium alginate and pectin. Preferably, the texturizing agent is selected from the group consisting of phosphates, sodium tripolyphosphate, sodium hexametaphosphate and sodium pyrophosphate.

The nutritional composition according to the invention may further comprise at least one supplementary food component. Preferably, the supplementary food component is selected from the group consisting of vitamins, minerals, fibers, vegetable oil, fatty acids, amino acids, flavoring agents, colorants, antioxidants, sweeteners, flavor enhancers, acidifiers, preservatives, sequestrants, seasonings, sugar, flour, prebiotics, salt, water, and antimicrobials. Preferably, the vitamin is selected from the group consisting of B vitamins including niacin, vitamin C and vitamin E. The flavoring agent is preferably selected from the group consisting of oleoresin and aquaresins. The colorant is preferably selected from the group consisting of curcumin, brilliant blue, tartrazine and ferrous gluconate. Preferably, the antioxidant is selected from the group consisting of nitrates, nitrites, ascorbyl palmitate and calcium ascorbate. Preferably, the sweetener is selected from the group consisting of sorbitol, alitame, aspartame, saccharin, calcium saccharin and corn syrup. The flavor enhancer is preferably selected from the group consisting of acetic acid, citric acid and fumaric acid. The acidifier is preferably lactic acid. The preservative is preferably selected from the group consisting of sodium nitrate, benzoic acid, sodium benzoate, tocopherols, ascorbic acid, niacin, riboflavin and thiamine. Preferably, the sequestrant is potassium gluconate. The seasoning is preferably selected from the group consisting of spices or oleoresins extracted from them, herbs, vegetables, essential oils, sodium nitrate, water, salt, sugar, and flavors. Preferably, the antimicrobial is selected from the group consisting of lactic acid, citric acid, acetic acid, sodium diacetate, acidified sodium chloride or calcium sulfate, activated lactoferrin, sodium or potassium lactate, or bacteriocins such as nisin.

### Use

As intended herein, “combined” or “in combination” means that a first compound as defined above, is administered at the same time than a second compound, either together, i.e. at the same administration site, or separately, or at different times, provided that the time period during which the first compound as defined above exerts its effects on the individual and the time period during which the second compound exerts its effects on the individual, at least partially intersect.

Preferably, at least two different compounds selected from the following pairs of compounds are used as defined above:

TABLE B

| | I.11 | I.12 | II.1 | II.2 | II.3 | II.4 | II.5 | II.6 | III.1 | III.2 | III.3 | III.4 |
|---|---|---|---|---|---|---|---|---|---|---|---|---|
| I.1 | X | X | X | X | X | X | X | X | X | X | X | X |
| I.2 | X | X | X | X | X | X | X | X | X | X | X | X |
| I.3 | X | X | X | X | X | X | X | X | X | X | X | X |
| I.4 | X | X | X | X | X | X | X | X | X | X | X | X |
| I.5 | X | X | X | X | X | X | X | X | X | X | X | X |
| I.6 | X | X | X | X | X | X | X | X | X | X | X | X |
| I.7 | X | X | X | X | X | X | X | X | X | X | X | X |
| I.8 | X | X | X | X | X | X | X | X | X | X | X | X |
| I.9 | X | X | X | X | X | X | X | X | X | X | X | X |
| I.10 | X | X | X | X | X | X | X | X | X | X | X | X |

Preferably, in the use as defined above, in particular wherein at least two different compounds selected from the pairs defined in Table B are used, at least a third compound selected from the group consisting of the compounds of respective formulae I.1, I.2, I.3, I.4, I.5, I.6, I.7., I.8, I.9, I.10, I.11, I.12, II.1, II.2, II.3, II.4, II.5, II.6, III.1, III.2, III.3 and III.4 is used, provided the third compound is different from the first and second compounds.

Preferably, at least one compound of formula (I), (II) or (III) is used as defined above in combination with the first and second compounds, provided the third compound is different from the first and second compounds.

In a preferred embodiment, at least two different compounds, or at least three different compounds, respectively selected from:
a compound of formula (I.1), (I.2) or (I.3),
a compound of formula (II.1), and
a compound of formula (II.3) or (II.4)
   are used as defined above.

Most preferably:
a compound of formula (I.1), (I.2) or (I.3),
a compound of formula (II.1), and
a compound of formula (II.3) or (II.4),
   are used as defined above.

Even more preferably:
a compound of formula (I.1), in particular of formula (I.1a);
a compound of formula (II.1), and
a compound of formula (II.3), in particular of formula (II.3a),
   are used as defined above.

Preferably:
a compound of formula (I.4), in particular of formula (I.4a);
a compound of formula (I.6), in particular of formula (I.6a), and
a compound of formula (II.1) or of formula (II.5),
   are used as defined above.

Preferably:
a compound of formula (I.4), in particular of formula (I.4a);
a compound of formula (II.1), and
a compound of formula (II.5),
   are used as defined above.

As intended herein an anti-aging agent relates to an agent which prevents, relieves, attenuates, alleviates or treats the physical consequences of aging.

As intended herein an antioxidant agent relates to an agent which prevents, relieves, attenuates, alleviates or treats the physical consequences of oxidative stress.

The compositions and the combinations of compounds according to the invention are useful to alleviate at least one sign of skin aging.

As intended herein a “sign of skin aging” relates to a skin defect which occurs as consequence of a degradation of skin constituents due to chronic factors, such as mechanical, oxidative and/or photo stresses. In particular skin aging can be a consequence of chronological aging and/or photo-aging. “Chronological aging” relates to skin defects which occur as a consequence of oldness. “Photo-aging” relates to skin defects which occur as a consequence of skin exposition to light, and in particular to UV rays, more particularly UV-A or UV-B rays.

The at least one sign of skin aging may notably be at least one wrinkle or loss of skin elasticity. In particular the at least one sign of skin aging is at least one sign of facial skin aging.

The at least one sign of skin aging alleviated according to the invention is preferably at least one wrinkle, in particular at least one mimic wrinkle and/or mechanical fold, more particularly selected from the group consisting of forehead lines, frown lines, crow's feet wrinkles, bunny lines, purse-string wrinkles, nasolabial folds, tear troughs, marionette lines, mental crease and neck lines.

The invention will be further described by the following non-limiting figure and example.

### DESCRIPTION OF THE FIGURES

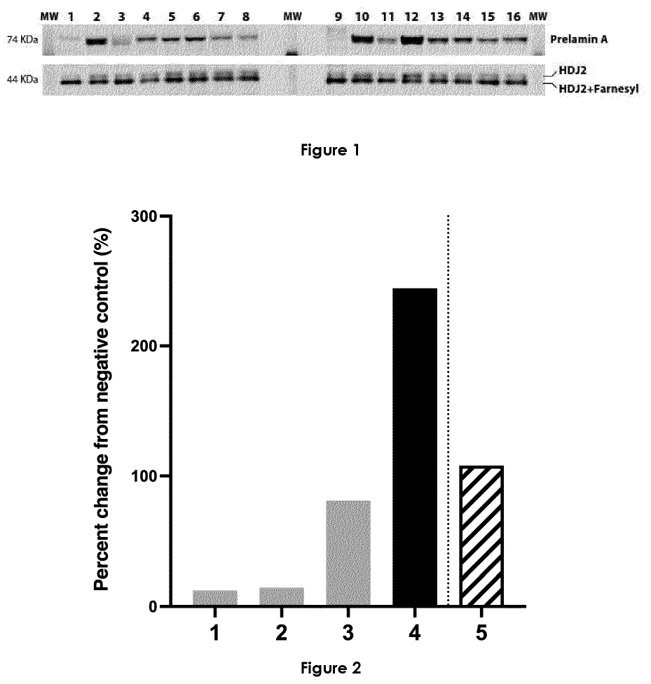

FIG. 1 represents a photograph of a western blot of proteins revealed by anti-lamin A/C, anti-prelamin A and anti-HDJ2 antibodies after having been extracted from human skin fibroblasts cultured in the presence of the following compounds:
1: DMSO (negative control)
2: Alendronate/Pravastatin (positive control)
3: Alendronate
4: Alendronate/Compound I.1a
5: Alendronate/Compound I.7a
6: Alendronate/Compound I.6a
7: Alendronate/Compound I.4a
8: Alendronate/Compound I.8a
9: DMSO
10: Alendronate/Pravastatin
11: Pravastatin
12: Pravastatin/Compound II.1
13. Pravastatin/Compound II.3a
14. Pravastatin/Compound II.2
15. Pravastatin/Compound I.12
16. Pravastatin/Compound II.5

FIG. 2 represents the LC3II/I protein ratio (vertical axis, in percent change from negative control) in human skin fibroblasts exposed to:
1: Compound I.1a alone
2: Compound II.1 alone
3: Compound II.3a alone
4: Combination compound I.1a+compound II.1+compound II.3a
Lane 5 represents the theoretical expected effect observed in case of additive effect of the compounds (addition of the respective effects of compound I.1a alone, compound II.1 alone and compound II.3a alone).

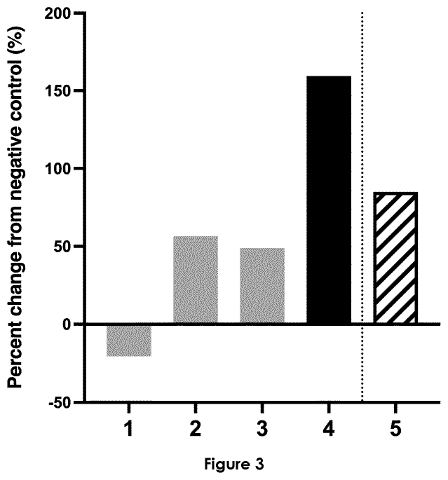

FIG. 3 represents the amount of Lamin B₁ (vertical axis, in percent change from negative control) in human skin fibroblasts exposed to:
1: Compound I.1a alone
2: Compound II.1 alone
3: Compound II.3a alone
4: Combination compound I.1a+compound II.1+compound II.3a
Lane 5 represents the theoretical expected effect observed in case of additive effect of the compounds (addition of the respective effects of compound I.1a alone, compound II.1 alone and compound II.3a alone).

### EXAMPLES

### Example 1

The topical administration of an alendronate-pravastatin composition on the face of human volunteers has shown an anti-skin aging effect in a double blind, randomized and placebo controlled comparative study, by reducing crow's feet wrinkles and restoring cheek volume. This effect is believed to be carried, at least in part, by the inhibition of the synthesis of farnesylpyrophosphate, the precursor of the farnesyl group which anchors the pro-aging factor progerin (Scaffidi & Misteli (2006) Science 312:1059-1063) to the nuclear envelop in cells from aged subjects.

Inhibition of progerin farnesylation has been shown to reduce progerin toxicity and its pro-aging consequences (Glynn & Glover (2005) Human Molecular Genetics 14:2959-2969; Varela et al. (2008) Nature Medicine 14:767-772). Farnesylated progerin sequesters and mislocalizes NRF2 within nuclear lamina, resulting in impaired NRF2 transcriptional activity which consequently increases chronic oxidative stress (Kubben et al. (2016). Cell 165, 1361-1374).

In the following, the inventors assessed the capacity of various tested compounds to substitute for either alendronate or pravastatin in the inhibition of the farnesylation of prelamin A and of HDJ2, which is indicative of anti-aging and antioxidant effects.

### Material and Methods

### Tested Compounds

The following compounds were tested:

| Structure | Name | CAS Number |
|---|---|---|
| 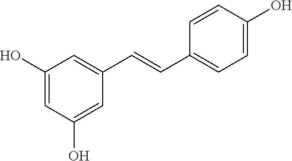 [SMILES: Oc1ccc(/C=C/c2cc(O)cc(O)c2)cc1] | I.1a | 501-36-0 |
| 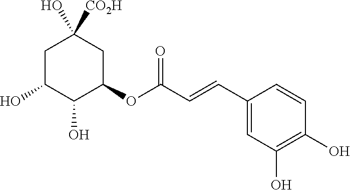 [SMILES: O=C(/C=C/c1ccc(O)c(O)c1)O[C@@H]1C[C@](O)(C(=O)O)C[C@@H](O)[C@H]1O] | I.4a | 327-97-9 |
| 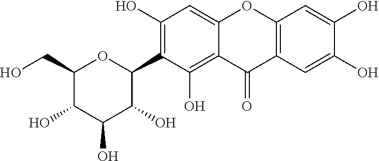 [SMILES: O=c1c2cc(O)c(O)cc2oc2cc(O)c([C@@H]3O[C@H](CO)[C@@H](O)[C@H](O)[C@H]3O)c(O)c12] | I.6a | 4773-96-0 |
| 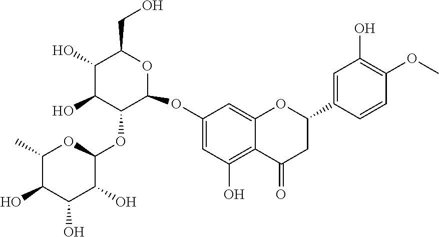 | I.7a | 13241-33-3 |
| 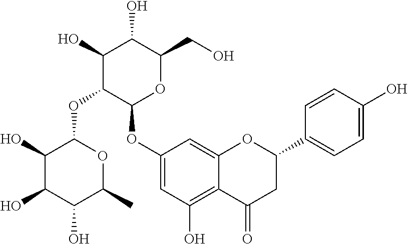 [SMILES: C[C@@H]1O[C@@H](O[C@H]2[C@H](Oc3cc(O)c4c(c3)O[C@H](c3ccc(O)cc3)CC4=O)O[C@H](CO)[C@@H](O)[C@@H]2O)[C@H](O)[C@H](O)[C@H]1O] | I.8a | 10236-47-2 |
| 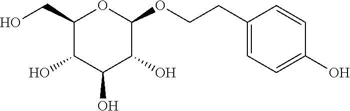 [SMILES: OC[C@H]1O[C@@H](OCCc2ccc(O)cc2)[C@H](O)[C@@H](O)[C@@H]1O] | I.12a | 10338-51-9 |
| 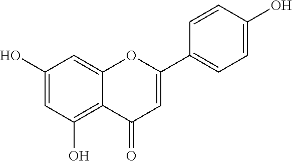 [SMILES: O=c1cc(-c2ccc(O)cc2)oc2cc(O)cc(O)c12] | II.1 | 520-36-5 |
| 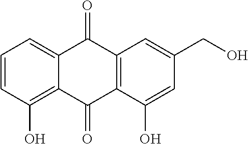 [SMILES: O=C1c2cccc(O)c2C(=O)c2c(O)cc(CO)cc21] | II.2 | 481-72-1 |
| 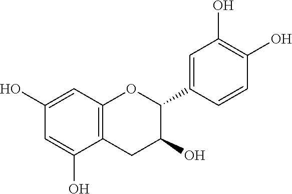 [SMILES: Oc1cc(O)c2c(c1)O[C@H](c1ccc(O)c(O)c1)[C@@H](O)C2] | II.3a | 154-23-4 |
| 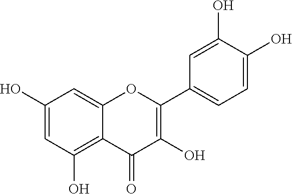 [SMILES: O=c1c(O)c(-c2ccc(O)c(O)c2)oc2cc(O)cc(O)c12] | II.5 | 117-39-5 |

The tested compounds were obtained from Euromedex, Souffelweyersheim, France for I.1a and Sigma, Saint-Quentin Fallavier, France for I.4a, I.6a, I.7a, I.8a, I.12a, II.1, II.2, II.3a and II.5. The tested compounds were diluted in DMSO.

Alendronate and pravastatin were obtained from AbMole/Euromedex, Souffelweyersheim, France and Alsachim, Illkirch-Graffenstaden, France respectively. Alendronate and pravastatin were diluted in H₂O.

### Cell Culture

Control Human fibroblast from skin (AG13334) were obtained from Coriell Institute. Cells were maintained in culture according to supplier information at 37° C. in a humidified atmosphere containing 5% CO₂.

Fibroblasts were cultured 72 h without culture-media renewal.

To assess cell viability, cells were treated 72 h with the tested compounds, with concentrations ranging from 1 pM to 1000 μM. Each tested compound was combined with Alendronate (10 μM) or Pravastatin (10 μM).

To assess efficacy, cells were treated by a combination of Alendronate or Pravastatin (10 μM) with the tested compounds at their highest non-toxic doses or half of this concentration (Compound I.1a, 50 μM; Compound I.7a, 500 M; Compound I.6a, 500 μM; Compound I.4a, 500 μM; Compound I.8a, 1000 μM; Compound II.1, 60 μM; Compound II.3a, 500 μM; Compound II.2, 30 μM; Compound I.12a 500 μM; Compound II.5, 250 μM).

The following combinations were tested:
1: DMSO (negative control)
2: Alendronate/Pravastatin (positive control)
3: Alendronate
4: Alendronate/Compound I.1a
5: Alendronate/Compound I.7a
6: Alendronate/Compound I.6a
7: Alendronate/Compound I.4a
8: Alendronate/Compound I.8a
9: DMSO
10: Alendronate/Pravastatin
11: Pravastatin
12: Pravastatin/Compound II.1
13. Pravastatin/Compound II.3a
14. Pravastatin/Compound II.2
15. Pravastatin/Compound I.12
16. Pravastatin/Compound II.5

Ritonavir (4 μM) and Lopinavir (20 μM) were added in all tested conditions to improve the lower limit of detection of prenylated prelamin A in western blots.

### Cell Viability and Assessment of Cell-Toxicity

Assays were carried out in 96-well microplates. After 72 hours of treatment, the cells were washed once with 100 μL DPBS (no calcium, no magnesium). Then 100 μl of PrestoBlue solution (Life Technologies) diluted at 10% in DPBS was added to each well. Plates were incubated at 37° C. for 30 minutes. The fluorescence intensity was measured by multiwell plate reader (Glomax microplate reader, Promega, Charbonnières les Bains, France) using green filter (Excitation 525 nm/emission 580-640 nm). Fluorescence intensities values were analysed in Prism Software (GraphPad, San Diego, CA).

### Protein Extraction

Total fibroblast proteins were extracted after 72 hours treatment in 100 μl of NP40 (Invitrogen) with 1× protease and phosphatase inhibitor cocktail (Life Technologies). Lysates were incubated on ice for 30 minutes, with vortexing at 10-minute intervals. Finally, they were sonicated four times (20 sec each) and then centrifuged at 13000 rpm for 10 minutes at 4° C.

Protein concentrations were determined with the BCA™ Protein Assay (Life Technologies).

### Antibodies

The following antibodies were used in this study: rabbit monoclonal anti-lamin A/C (ab108922, 1/1000, Abcam, Amsterdam, Netherlands), mouse monoclonal anti-prelamin A (MABT858, Millipore, Molsheim, France), mouse monoclonal anti-HDJ2 (MA5-12748, Thermoscientific, France). Secondary antibodies conjugated with IR-Dye 800CW or 680 were used according to the manufacturer's instructions (926-32213 and 926-68072, 1/5000, Li-COR Biosciences).

### Western Blotting

Protein lysates were separated on NuPAGE™ Novex™ 8% Bis-Tris Midi Protein Gels (Life Technologies) and transferred to Immobilon-FL PVDF membranes (Millipore, Molsheim, France). Membranes were blocked for one hour in 1:1 diluted blocking buffer for near fluorescent western blotting (Rockland, Le Perray, France). Blocked membranes were incubated with primary antibody anti-prelamin A overnight at 4° C., following which they were washed and incubated with conjugated secondary antibody for one hour at room temperature. Then, this step was repeated with primary antibodies for HDJ2 and LMNA/C, and conjugated secondary antibodies.

Bound antibodies were detected and analyzed on an Odyssey imaging system (Li-COR Biosciences, Bad Homburg, Germany) according to the manufacturer's instructions. Revert Protein Stain (Li-COR Biosciences) was used as a total protein loading control.

### Results

The results are shown in FIG. 1.

Prelamin A and unfarnesylated HDJ2 are absent in lanes 1 and 9, negative control(s) (DMSO) and essentially absent in lanes 3 for alendronate and 11 for pravastatin alone.

In contrast, a strong band can be seen for prelamin A for the alendronate/pravastatin combination (positive control, lanes 2 and 10). Indeed, prelamin A accumulates due to the inhibition of farnesylation which prevents its maturation to lamin A. A clear band can also be seen for unfarnesylated HDJ2.

Clear bands are also visible for prelamin A and for unfarnesylated HDJ2 for the combinations of the tested compounds I.1a, I.4a, I.6a, I.7a and I.8a (lanes 4, 7, 6, 5 and 8 respectively) with alendronate and of compounds I.12, II.1, II.2, II.3a and II.5 (lanes 15, 12, 14, 13 and 16 respectively) with pravastatin, which indicates an inhibition of farnesylation and therefore an anti-aging effect.

This also indicates that combining compounds I.1a, I.4a, I.6a, I.7a and I.8a with compounds I.12, II.1, II.2, II.3a and II.5 should also yield an inhibition of farnesylation and anti-aging and an antioxidant effects.

### Example 2

Autophagy is an evolutionarily conserved, lysosome-dependent catabolic process whereby cytoplasmic components, including damaged organelles, protein aggregates and lipid droplets, are degraded and their components recycled. Autophagy has an essential role in maintaining cellular homeostasis in response to intracellular stress; however, the efficiency of autophagy declines with age (Kitada et al. (2021) Nat Rev Endocrinol. 17:647-661). Conversely, autophagy activation promotes longevity (Hansen et al. (2018) Nat Rev Mol Cell Biol. 9:579-593).

Autophagy activation can be monitored through the conversion of microtubule-associated protein light chain 3 I (LC3I) to LC3II, in particular by measuring the LC3II/LC3-I ratio (also named LC3II/I ratio) by immunoblot analysis, whereby an increased ratio indicates an increased autophagy (Klionsky et al. (2021) Autophagy 17:1-382).

### Material and Methods

### Cell Culture

Control Human fibroblast from skin (AG13334) were obtained from Coriell Institute. Cells were maintained in culture according to supplier information at 37° C. in a humidified atmosphere containing 5% CO₂.

Fibroblasts were cultured 72 h without culture-media renewal.

To assess efficacy, cells were treated 72 h with the tested molecules alone or in combination (Compound I.1a, 50 μM; Compound II.1, 60 μM; Compound II.3a, 500 μM). As for the detection of prenylated prelamin A, Ritonavir (4 μM) and Lopinavir (20 μM) were added in all tested conditions and negative control.

The following conditions were tested:
1: Compound I.1a alone
2: Compound II.1 alone
3: Compound II.3a alone
4: Combination Compound I.1a+Compound II.1+Compound II.3a
5: Theoretical additive compounds effect (Compound I.1a+Compound II.1+Compound II.3)

### Protein Extraction

Total fibroblast proteins were extracted after 72 hours treatment in 100 μl of NP40 (Invitrogen) with 1× protease and phosphatase inhibitor cocktail (Life Technologies). Lysates were incubated on ice for 30 minutes, with vortexing at 10-minute intervals. Finally, they were sonicated four times (20 sec each) and then centrifuged at 10000 g for 10 minutes at 4° C.

Protein concentrations were determined with the Pierce™ BCA Protein Assay (Life Technologies).

### Antibodies

The following antibodies were used in this study: mouse monoclonal anti-LC3B (sc271625, 1/200, Santa Cruz, Heidelberg, Germany). Secondary antibodies conjugated with IR-Dye 800CW were used according to the manufacturer's instructions (926-32212, 1/5000, Li-COR Biosciences).

### Western Blotting

Protein lysates were separated on NuPAGE™ Novex™ 8% Bis-Tris Midi Protein Gels (Life Technologies) and transferred to Immobilon-FL PVDF membranes (Millipore, Molsheim, France). Membranes were blocked for one hour in 1:1 diluted blocking buffer for near fluorescent western blotting (Rockland, Le Perray, France). Blocked membranes were incubated with primary antibody anti-LC3B overnight at 4° C., following which they were washed and incubated with conjugated secondary antibody for one hour at room temperature.

Bound antibodies were detected and analyzed on an Odyssey imaging system (Li-COR Biosciences, Bad Homburg, Germany) according to the manufacturer's instructions. LC3II and LC3I were detected.

Results were expressed as percent change from negative control of LC3II/I ratio.

### Results

The results are shown in FIG. 2.

In can be seen that the tested combination of compounds I.1a, II.1, and II.3a yields an increase in the LC3II/I ratio with respect to the negative control (DMSO) (lane 5) which is greater than the increase expected from the addition of the respective increases in the LC3II/I ratio yielded by each compound alone (lane 6).

Accordingly, the effect of the tested combination of compounds I.1a, II.1, and II.3a on the activation of autophagy in human skin fibroblasts is supra-additive, i.e. it is synergistic.

The tested combination is therefore expected to exert anti-aging effects on the skin.

### Example 3

Depletion of Lamin B₁ from the nuclear envelope is an important event during senescence. Lamin B₁ is crucial to maintain the structural integrity of the nucleus. However, this integrity is lost in senescent cells, which allows the release of cytoplasmic chromatin fragments outside the nucleus, which is believed to fuel the SASP through the cGAS/STING pathway (Gonzalez-Gualda et al. (2021) The FEBS Journal 288:56-80). The loss of Lamin B₁ is thus a hallmark of senescent cells (Wang et al. (2017) Sci Rep. 7:15678).

The loss of Lamin B1 can be detected immunoassayed and detected by imaging or immunoblotting.

### Material and Methods

### Cell Culture

Control Human fibroblast from skin (AG13334) were obtained from Coriell Institute. Cells were maintained in culture according to supplier information at 37° C. in a humidified atmosphere containing 5% CO₂.

Fibroblasts were cultured 72 h without culture-media renewal.

To assess efficacy, cells were treated 72 h with the tested molecules alone or in combination (Compound I.1a, 50 μM; Compound II.1, 60 M; Compound II.3a, 500 μM). As for the detection of prenylated prelamin A, Ritonavir (4 μM) and Lopinavir (20 μM) were added in all tested conditions and negative control.

The following conditions were tested:
1: Compound I.1a alone
2: Compound II.1 alone
3: Compound II.3a alone
4: Combination Compound I.1a+Compound II.1+Compound II.3a
5: Theoretical additive compounds effect (Compound I.1a+Compound II.1+Compound II.3a)

### Protein Extraction

Total fibroblast proteins were extracted after 72 hours treatment in 100 μL of NP40 (Invitrogen) with 1× protease and phosphatase inhibitor cocktail (Life Technologies). Lysates were incubated on ice for 30 minutes, with vortexing at 10-minute intervals. Finally, they were sonicated four times (20 sec each) and then centrifuged at 10000 g for 10 minutes at 4° C.

Protein concentrations were determined with the Pierce™ BCA Protein Assay (Life Technologies).

### Antibodies

The following antibodies were used in this study: rabbit polyclonal anti-laminB1 (ab16048, 1/1000, Abcam, Amsterdam, Netherlands). Secondary antibodies conjugated with IR-Dye 680RD were used according to the manufacturer's instructions (926-68073, 1/5000, Li-COR Biosciences).

### Western Blotting

Protein lysates were separated on NuPAGE™ Novex™ 8% Bis-Tris Midi Protein Gels (Life Technologies) and transferred to Immobilon-FL PVDF membranes (Millipore, Molsheim, France). Membranes were blocked for one hour in 1:1 diluted blocking buffer for near fluorescent western blotting (Rockland, Le Perray, France). Blocked membranes were incubated with primary antibody anti-lamin B₁ overnight at 4° C., following which they were washed and incubated with conjugated secondary antibody for one hour at room temperature.

Bound antibodies were detected and analyzed on an Odyssey imaging system (Li-COR Biosciences, Bad Homburg, Germany) according to the manufacturer's instructions. All bands detected with anti LaminB1 were considered as specific (KO validated antibody reference). Revert Protein Stain (Li-COR Biosciences) was used as a total protein loading control.

Results were expressed as percent change from negative control of Lamin B1.

### Results

The results are shown in FIG. 3.

In can be seen that the tested combination of compounds I.1a, II.1, and II.3a yields an increase in the amount of Lamin B₁ with respect to the negative control (DMSO) (lane 5) which is greater than the increase expected from the addition of the respective increases in the amount of LaminB1 yielded by each compound alone (lane 6).

Accordingly, the effect of the tested combination of compounds I.1a, II.1, and II.3a on the reversal of senescence signs in human skin fibroblasts is supra-additive, i.e. it is synergistic.

The tested combination is therefore expected to exert anti-aging effects on the skin.

1. A composition comprising at least one first compound of the following formula (I), or a polymer of the first compound: 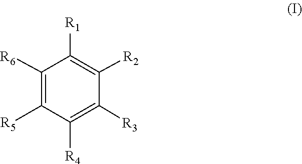
wherein:
R₁ and R₄, identical or different, represent —H, —OH, or —OCH₃,
R₂ and R₃, identical or different, represent —H, —OH, —OSO₃H, —O-hexoside, or —O—R₃₁, —O—CO—R₃₁ or —CH₂O—CO—R₃₁, wherein R₃₁ represents a saturated or unsaturated, linear or branched, aliphatic group comprising from 1 to 30 carbon atoms, optionally substituted by at least one ═O, —OH or —COOH group, or R₂ and R₃ represent together a group of the following formula (Ia) or (Ib): 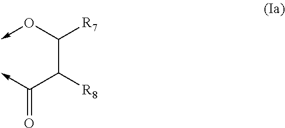
wherein:
R₇ represents —H, —OH, —OCH₃ or an aryl group comprising from 5 to 8 carbon atoms optionally substituted by at least one-OH group or oxyalkyl group comprising from 1 to 5 carbon atoms,
R₈ represents —H, —OH or —OCH₃, 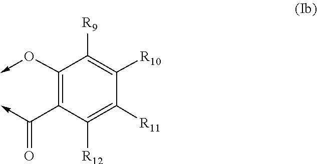
wherein R₉, R₁₀, R₁₁ et R₁₂, identical or different, represent —H, —OH or —OCH₃,
one of R₅ and R₆ represents —H, —OH or —OCH₃ and the other represents a saturated or unsaturated, linear or branched, aliphatic group comprising from 1 to 10 carbon atoms, optionally substituted by at least one ═O, —OH or —COOH group, or a group of the following formula (Ic): 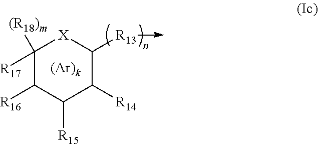
wherein
n=0 or 1,
k=0 or 1, when k=1 the above cycle is aromatic (Ar) and when k=0 the above cycle is not aromatic,
m=1 when k=0 and m=0 when k=1,
X represents —O—, —CH— or —CH₂—,
R₁₃ represents —O—, a group R₂₃ or a group —O—R₂₃— wherein R₂₃ represents a saturated or unsaturated, linear or branched, aliphatic group comprising from 1 to 5 carbon atoms, optionally substituted by at least one ═O group,
R₁₄ represents —H, —OH, —OCH₃ or a group of the following formula (Id): 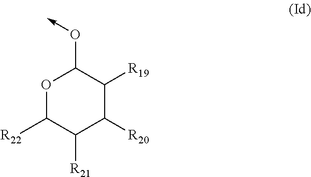
wherein R₁₉, R₂₀, R₂₁ and R₂₂, identical or different, represent —OH, —CH₂OH, an alkyl group comprising from 1 to 5 carbon atoms or an oxyalkyl group comprising from 1 to 5 carbon atoms,
R₁₅, R₁₆, R₁₇ and R₁₈, identical or different, represent —H, —OH, —OSO₃H, —CH₂OH, —COOH, —O-glucuronide, —O—R₃₀, —O—CO—R₃₀ or —CH₂O—CO—R₃₀, wherein R₃₀ represent a saturated or unsaturated, linear or branched, aliphatic group comprising from 1 to 30 carbon atoms, optionally substituted by at least one ═O, —OH or —COOH group,
and at least one second compound of formula (I), of the following formula (II) or of the following formula (III), the second compound of formula (I) being different from the first compound of formula (I): 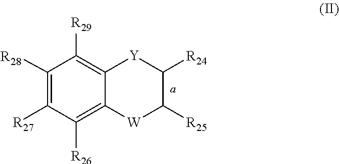
wherein:
a represents a single bond or a double bond,
Y represents —O— or —CO—,
W represents —CH₂— or —CO—,
R₂₄ represents an aryl group comprising from 5 to 8 carbon atoms, optionally substituted by at least one-OH, —OCH₃, —O-hexoside or —O-glucuronide group, R₂₅ represents —H, —OH, or an oxycarbonylaryl group comprising from 6 to 9 carbon atoms, optionally substituted by at least one-OH, —OCH₃, —O-hexoside or O-glucuronide group, or R₂₄ and R₂₅ represent together, with the carbon atoms to which they are attached and a representing a double bond, an aryl group comprising 6 carbon atoms, optionally substituted by at least one-OH or —CH₂OH group,
R₂₆, R₂₇ and R₂₈, identical or different, represent —H, —OH, —OCH₃, —O-hexoside or O-glucuronide,
R₂₉ represents —H, —OH or a group of the following formula (IIa): 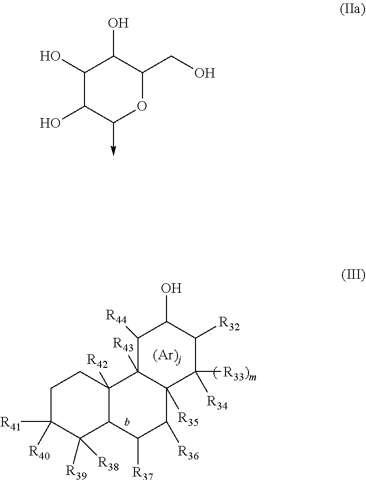
wherein:
j=0 or 1, when j=1 the above cycle is aromatic (Ar) and when j=0 the above cycle is not aromatic,
m=1 when j=0 and m=0 when j=1,
when j=1, R₃₂ represents an alkyl group having from 1 to 5 carbon atoms and when j=0, R₃₂ together with R₃₃ represent a group of the following formula (IIIa): 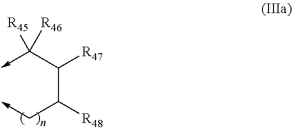
wherein:
n=0 or 1,
R₄₅ represents —H or —CH₃,
R₄₆ represents —CH₂OH or a group of the following formula (IIIb) or (IIIc): 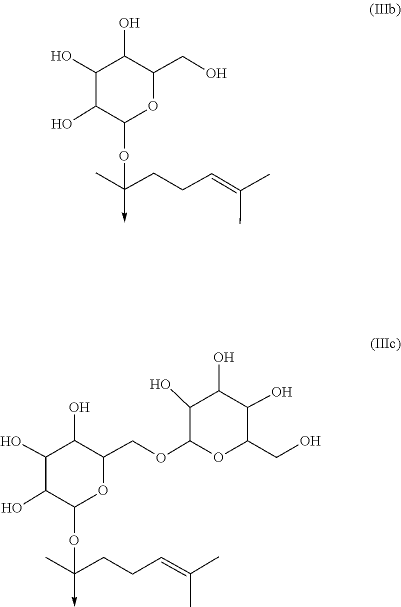
R₄₇ and R₄₈, identical or different, represent —H or —OH
R₃₄, R₃₅, R₃₈, and R₄₃, identical or different, represent —H or —CH₃,
R₃₆ represents —H, R₄₂ represents —CH₃, or together R₃₆ and R₄₂ represent a group of formula —O—CO—,
R₃₇ represents —H or —O-hexoside,
b represents a single bond or a double bond,
R₃₉ represents —CH₃, R₄₀ represents —H, or together R₃₉ and R₄₀ represent a group of the following formula (IIId): 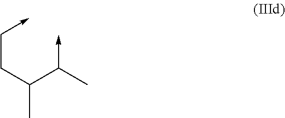
R₄₁ represents —H, —OH or a group of the following formula (IIIe) or (IIIf): 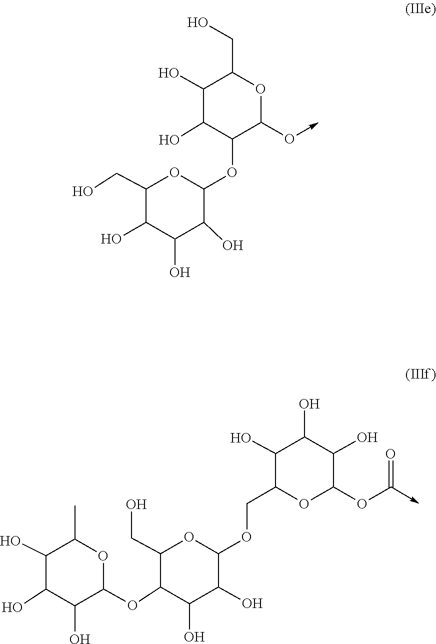
R₄₄ represents —H or —OH,
or cosmetically or nutritionally acceptable salts or esters thereof.

2. The composition according to claim 1, wherein the first compound is selected from the group consisting of the compounds of the following formulae: 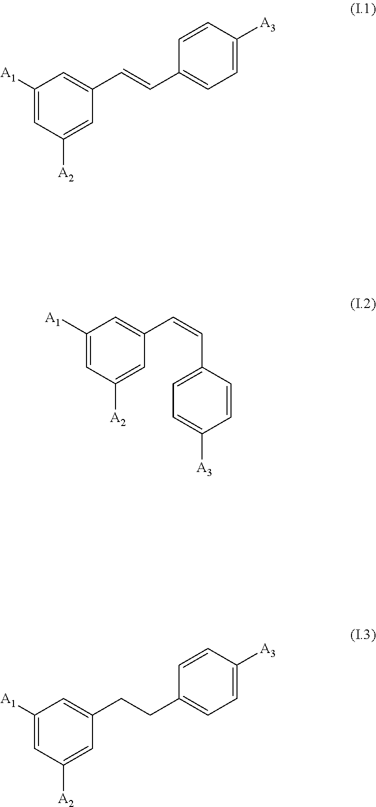
wherein A₁, A₂ and A₃, identical or different, represent —OH, —OSO₃H, —O-glucuronide, or —O—CO-A₄, wherein A₄ represents an aliphatic group comprising from 1 to 30 carbon atoms, saturated or unsaturated, linear or branched, optionally substituted by at least one ═O, —OH or —COOH group,

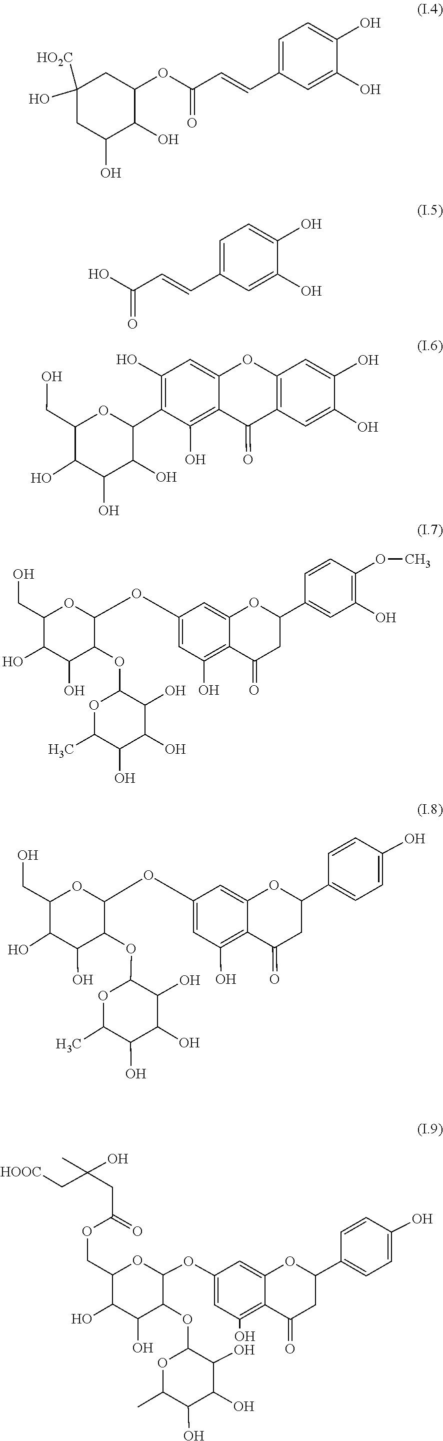

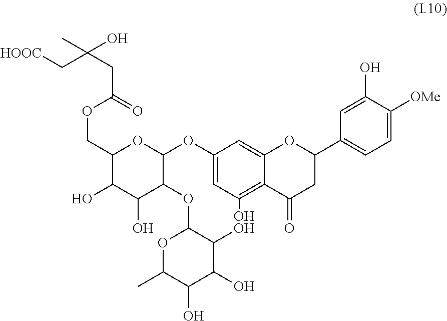 [SMILES: COc1ccc(C2CC(=O)c3c(O)cc(OC4OC(COC(=O)CC(C)(O)CC(=O)O)C(O)C(O)C4OC4OC(C)C(O)C(O)C4O)cc3O2)cc1O]

3. The composition according to claim 1, wherein the second compound is selected from the group consisting of the compounds of the following formulae: 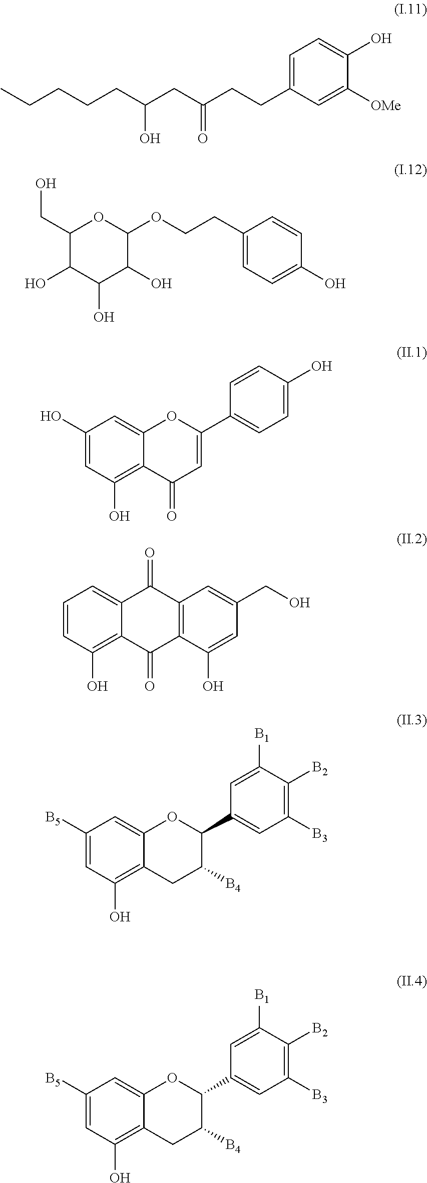
wherein:
B₁ represents —OH, —OCH₃ or —O-glucuronide,
B₂ represents —OH or —OCH₃,
B₃ represents —H or —OH,
B₄ represents —OH, —O-glucuronide or a group of the following formula: 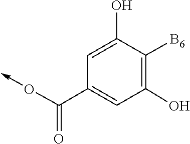
wherein B₆ represents —OH, —OCH₃ or —O-glucuronide,
B₅ represents —OH, —O-hexoside or —O-glucuronide,

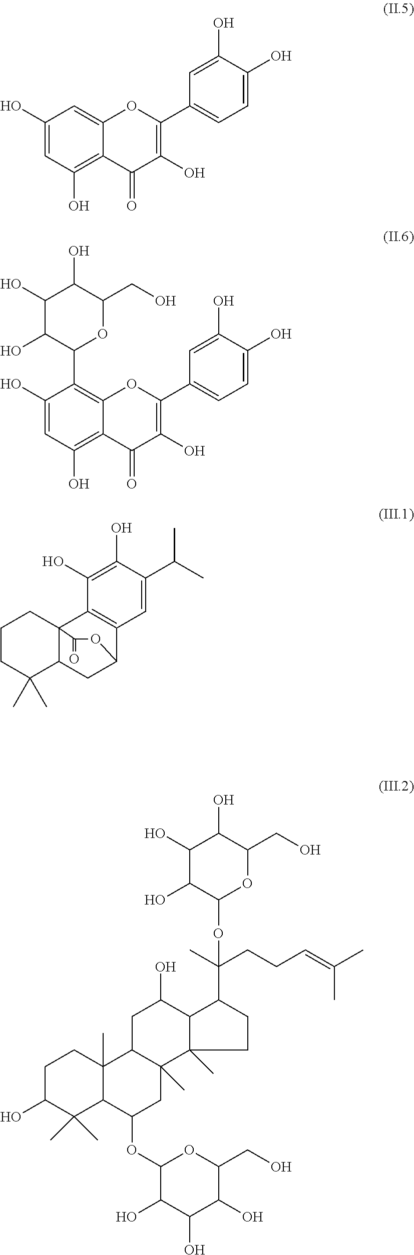

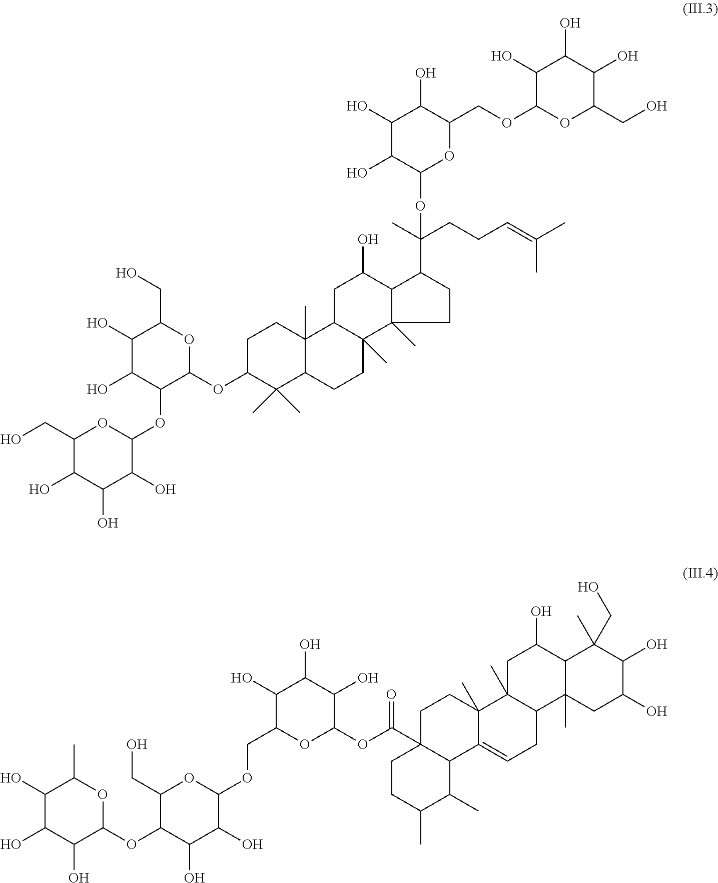 [SMILES: CC(C)=CCCC(C)(OC1OC(COC2OC(CO)C(O)C(O)C2O)C(O)C(O)C1O)C1CCC2(C)C1C(O)CC1C3(C)CCC(OC4OC(CO)C(O)C(O)C4OC4OC(CO)C(O)C(O)C4O)C(C)(C)C3CCC12C ; CC1CCC2(C(=O)OC3OC(COC4OC(CO)C(OC5OC(C)C(O)C(O)C5O)C(O)C4O)C(O)C(O)C3O)CCC3(C)C(=CCC4C5(C)CC(O)C(O)C(C)(CO)C5C(O)CC43C)C2C1C]

4. The composition according to claim 1, further comprising at least one third compound of formula (I), (II) or (III), provided the third compound is different from the first and second compounds.

5. The composition according to claim 1, comprising at least two different compounds, or at least three different compounds, selected respectively from:
a compound of formula (I.1), (I.2) or (I.3),
a compound of formula (II.1), and
a compound of formula (II.3) or (II.4).

6. The composition according to claim 1, wherein the compounds of formula (I), (II) and (III) are comprised in at least one vegetal extract.

7. The composition according to claim 1, further comprising at least one cosmetically or nutritionally acceptable carrier or excipient.

8. A method of preventing, attenuating, alleviating, or treating skin aging, comprising administering a cosmetic agent to the skin or a human or animal food supplement to the diet, wherein the cosmetic agent or human or animal food supplement comprises the composition of claim 1.

9. The method of claim 8, wherein at least one sign of skin aging is alleviated.

10. The method of claim 8, wherein the cosmetic agent or a human or animal food supplement composition comprises at least one first compound of formula (I), or of at least one polymer of the first compound, in combination with at least one second compound of formula (I), of formula (II), or of formula (III), the second compound of formula (I) being different from the first compound of formula (I).

11. The method of claim 8, wherein the cosmetic agent or a human or animal food supplement composition comprises at least one first compound of formula (I), or of at least one polymer of the first compound, in combination with at least one second compound of formula (I), of formula (II), or of formula (III), the second compound of formula (I) being different from the first compound of formula (I), to alleviate at least one sign of skin aging.

12. The method of claim 8, wherein the cosmetic agent or a human or animal food supplement composition comprises a first compound selected from the group consisting of the compounds of the following formulae: 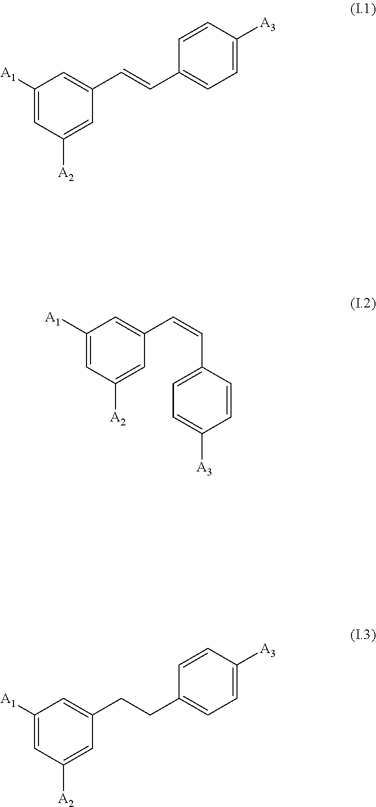
wherein A₁, A₂ and A₃, identical or different, represent —OH, —OSO₃H, —O-glucuronide, or —O—CO-A₄, wherein A₄ represents an aliphatic group comprising from 1 to 30 carbon atoms, saturated or unsaturated, linear or branched, optionally substituted by at least one ═O, —OH or —COOH group,

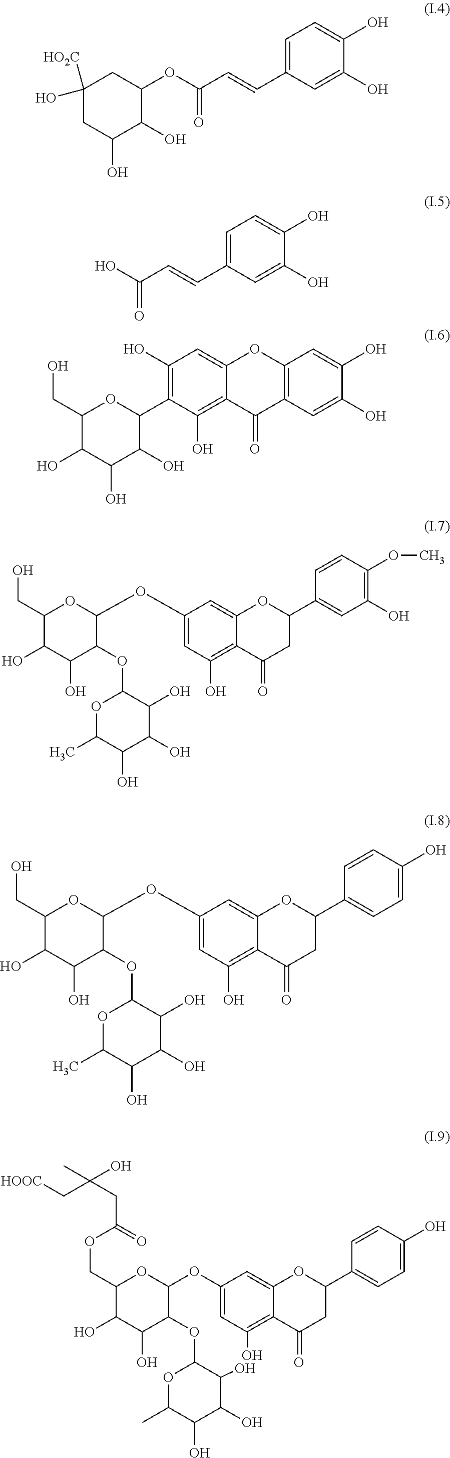

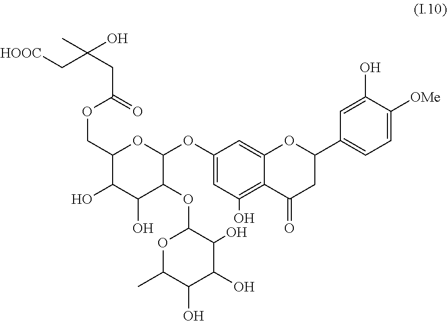 [SMILES: COc1ccc(C2CC(=O)c3c(O)cc(OC4OC(COC(=O)CC(C)(O)CC(=O)O)C(O)C(O)C4OC4OC(C)C(O)C(O)C4O)cc3O2)cc1O]

13. The method of claim 8, wherein the cosmetic agent or a human or animal food supplement composition comprises a second compound selected from the group consisting of the compounds of the following formulae: 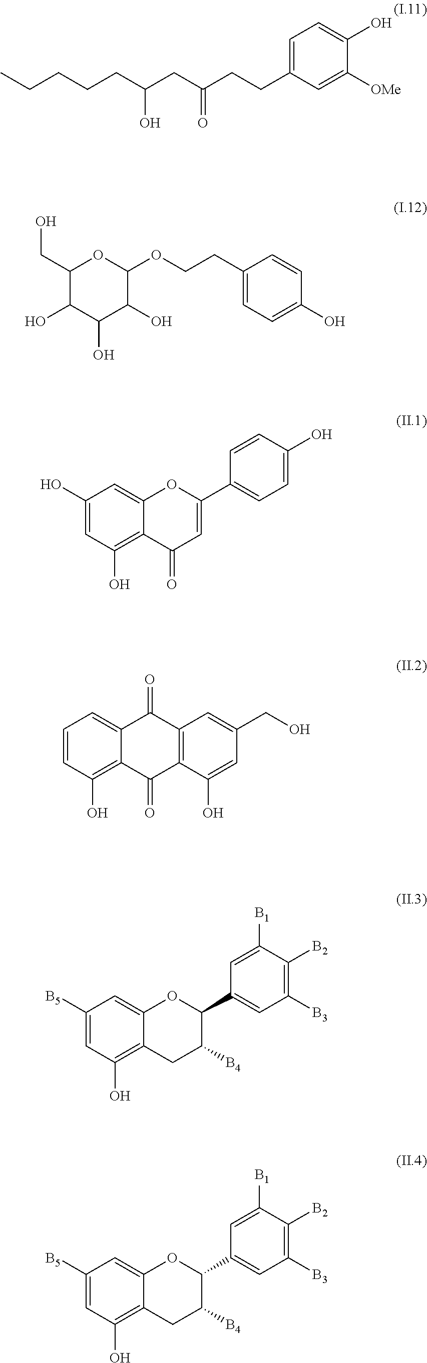
wherein:
B₁ represents —OH, —OCH₃ or —O-glucuronide,
B₂ represents —OH or —OCH₃,
B₃ represents —H or —OH,
B₄ represents —OH, —O-glucuronide or a group of the following formula: 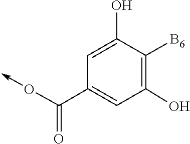
wherein B₆ represents —OH, —OCH₃ or —O-glucuronide,
B₅ represents —OH, —O-hexoside or —O-glucuronide,

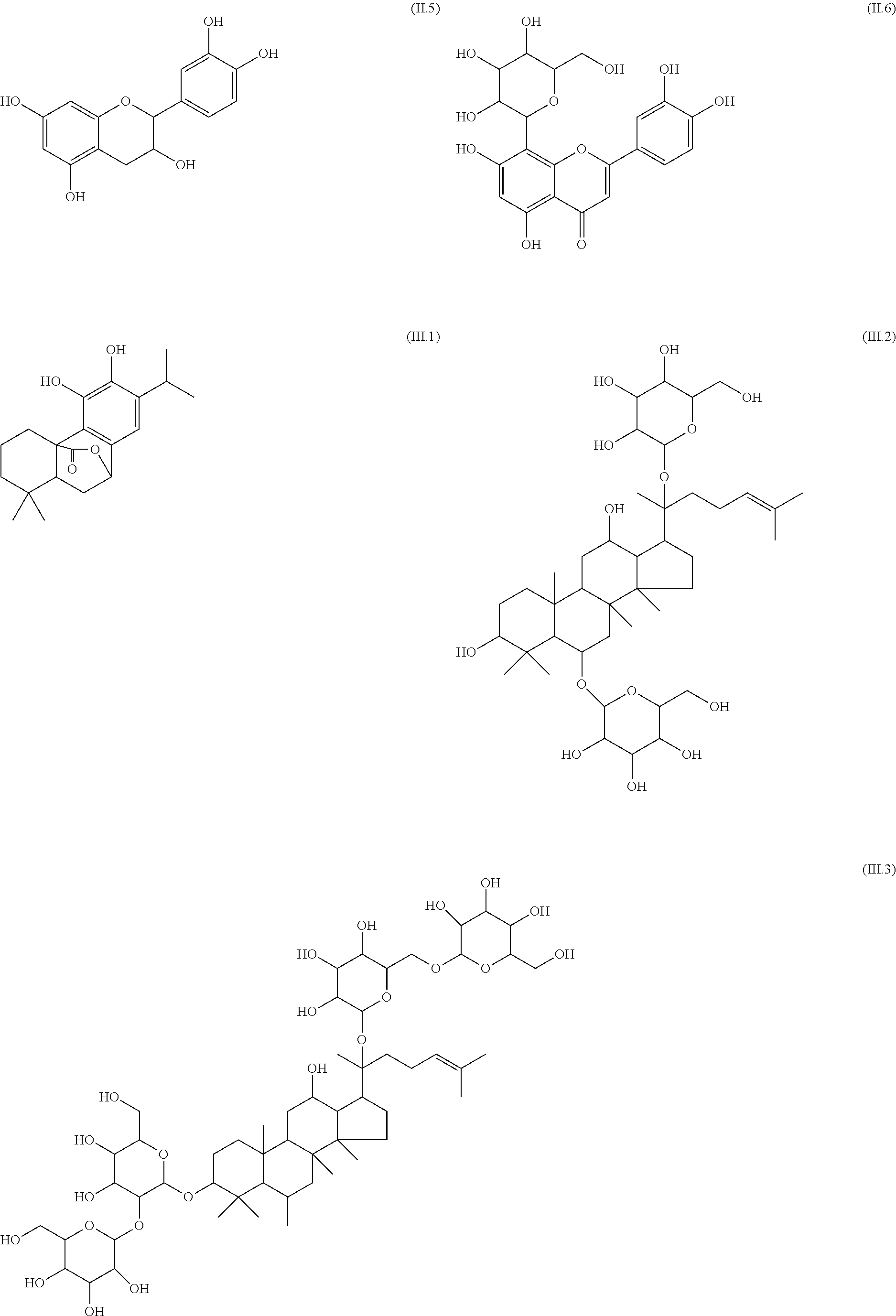 [SMILES: Oc1cc(O)c2c(c1)OC(c1ccc(O)c(O)c1)C(O)C2 ; O=c1cc(-c2ccc(O)c(O)c2)oc2c(C3OC(CO)C(O)C(O)C3O)c(O)cc(O)c12 ; CC(C)c1cc2c(c(O)c1O)C13CCCC(C)(C)C1CC2OC3=O ; CC(C)=CCCC(C)(OC1OC(CO)C(O)C(O)C1O)C1CCC2(C)C1C(O)CC1C3(C)CCC(O)C(C)(C)C3C(OC3OC(CO)C(O)C(O)C3O)CC12C ; CC(C)=CCCC(C)(OC1OC(COC2OC(CO)C(O)C(O)C2O)C(O)C(O)C1O)C1CCC2(C)C1C(O)CC1C3(C)CCC(OC4OC(CO)C(O)C(O)C4OC4OC(CO)C(O)C(O)C4O)C(C)(C)C3C(C)CC12C]

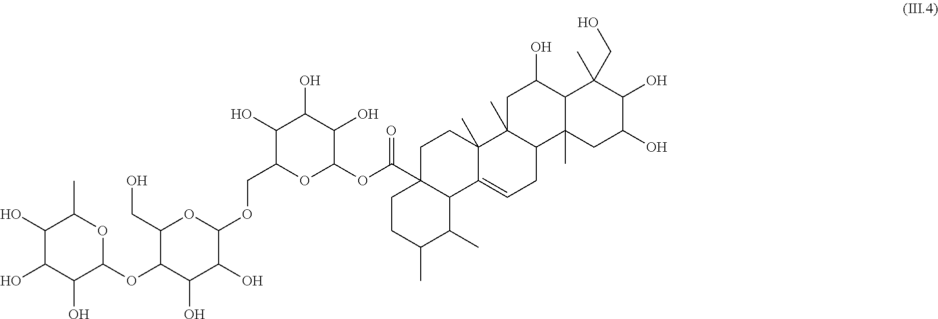 [SMILES: CC1CCC2(C(=O)OC3OC(COC4OC(CO)C(OC5OC(C)C(O)C(O)C5O)C(O)C4O)C(O)C(O)C3O)CCC3(C)C(=CCC4C5(C)CC(O)C(O)C(C)(CO)C5C(O)CC43C)C2C1C]

14. The method of claim 8, wherein the cosmetic agent or a human or animal food supplement composition comprises at least one compound of formula (I), (II) or (III) in combination with the first and second compounds, provided the third compound is different from the first and second compounds.

15. The method of claim 8, wherein the cosmetic agent or a human or animal food supplement composition comprises at least two different compounds, or at least three different compounds, respectively selected from:
a compound of formula (I.1), (I.2) or (I.3),
a compound of formula (II.1), and
a compound of formula (II.3) or (II.4).

16. The method of claim 9, wherein the at least one sign of skin aging that is alleviated is selected from the group consisting of at least one of forehead lines, frown lines, crow's feet wrinkles, bunny lines, purse-string wrinkles, nasolabial folds, tear troughs, marionette lines, mental crease and neck lines.
